(19) United States
(12) Patent Application Publication
(10) Pub. No.: US 2025/0179029 A1
(43) Pub. Date: Jun. 5, 2025

(54) DEUTERATED RETINOIDAL COMPOUNDS AND SYNTHESIS AND USES THEREOF

(71) Applicant: UNIVERSITY OF MARYLAND, BALTIMORE, Baltimore, MD (US)

(72) Inventors: Vincent C.O. NJAR, Glen Burnie, MD (US); Purushottamachar PURANIK, Urbana, MD (US); Elizabeth THOMAS, Baltimore, MD (US); Retheesh SULOCHANA THANKAN, Baltimore, MD (US)

(73) Assignee: UNIVERSITY OF MARYLAND, BALTIMORE, Baltimore, MD (US)

(21) Appl. No.: 18/842,113

(22) PCT Filed: Mar. 3, 2023
(86) PCT No.: PCT/US2023/063647

§ 371 Date: Aug. 28, 2024

Related U.S. Application Data
(60) Provisional application No. 63/487,705, filed on Mar. 1, 2023.
(60) Provisional application No. 63/316,328, filed on Mar. 3, 2022.

(51) Int. Cl. C07D 233/64; A61K 9/00; A61K 31/4164; A61P 35/00; C07B 59/00

(52) U.S. Cl. CPC C07D 233/64; A61K 9/0053; A61K 31/4164; A61P 35/00; C07B 59/002; C07B 2200/05

(57) ABSTRACT

The present disclosure relates to novel deuterated retinoidal compounds and methods of preparing the deuterated retinoidal compounds. The present disclosure also provides pharmaceutical compositions including the deuterated retinoidal compounds, and uses of the deuterated retinoidal compounds to treat diseases including breast and prostate cancers.

### CROSS-REFERENCE TO RELATED APPLICATIONS

This application is based on, and claims priority to, U.S. provisional application Ser. No. 63/316,328, filed on Mar. 3, 2022, and U.S. provisional application Ser. No. 63/487,705, filed on Mar. 1, 2023, the entire contents of which are herein incorporated by reference.

### STATEMENT OF FEDERALLY SPONSORED RESEARCH AND DEVELOPMENT

This invention was made with government support under Grant Numbers CA129379 and CA224696 awarded by the National Institutes of Health. The government has certain rights in the invention.

### TECHNICAL FIELD

The present disclosure generally relates to novel deuterated retinoidal compounds that are useful as degraders of mitogen-activated protein kinase (MAPK)-interacting kinases 1 and 2 (Mnk1/2). The present disclosure also provides methods of preparing the deuterated retinoidal compounds, pharmaceutical compositions comprising the deuterated retinoidal compounds, and methods of using the deuterated retinoidal compounds in the treatment of various Mnk1/2-associated diseases.

### BACKGROUND

There continues to be a need for highly effective therapeutic agents for treating cancers that have a sufficiently large therapeutic window and that have no or limited toxicities or side effects. The present inventors have discovered novel deuterated retinoidal compounds with unique properties and biological activities, including modulation of Mnk1/2-eukaroytic translation initiation factor (eIF4E) and androgen receptor (AR) signaling pathways, that are capable of effecting treatment of a variety of cancers and diseases that depend on functional Mnk1/2, androgen receptor, and/or a splice variant of the androgen receptor for their pathogenesis, while limiting adverse side effects.

### BRIEF SUMMARY OF INVENTION

The present disclosure provides a compound of formula (A)

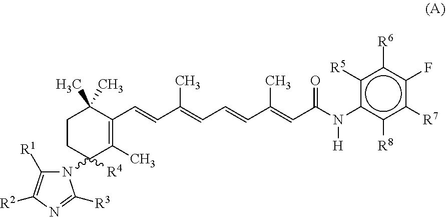

or a pharmaceutically acceptable salt thereof, wherein each of R¹ to R⁸ may be independent H or D, and at least one of R¹ to R⁸ is D. In some embodiments, R⁴ and at least one of R⁶ to R⁸ may be D.

In some embodiments, the compound may be at least one selected from the group consisting of:

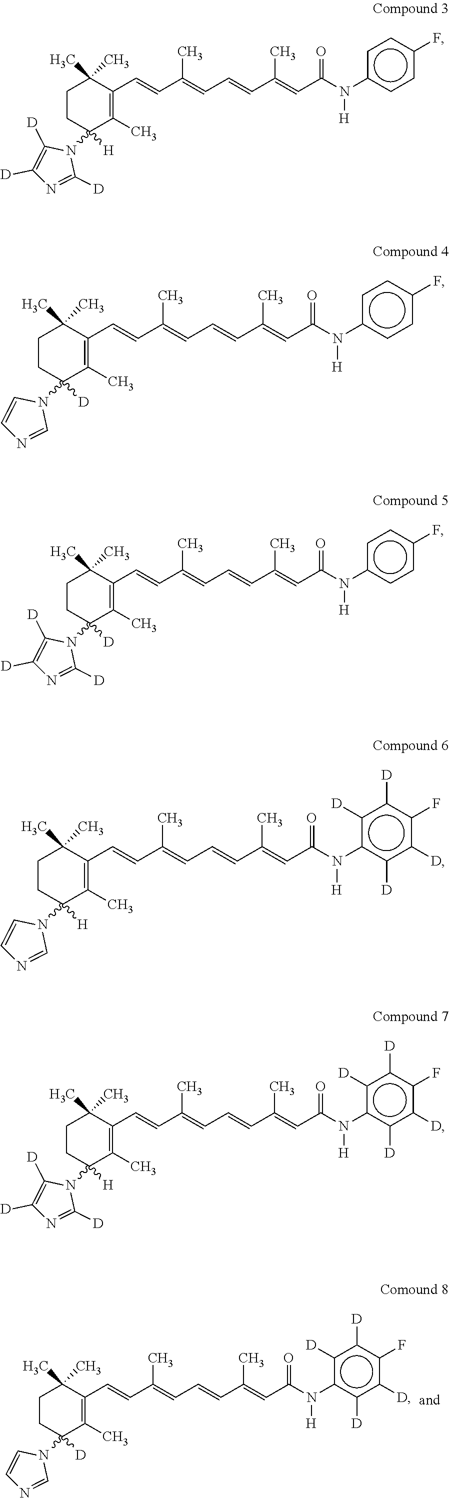

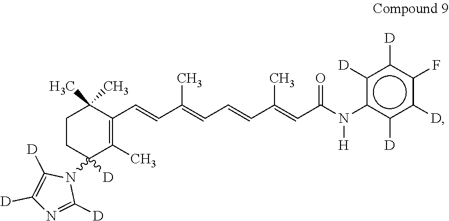

or a pharmaceutically acceptable salt thereof. In some embodiments, the compound may be at least one selected from the group consisting of:

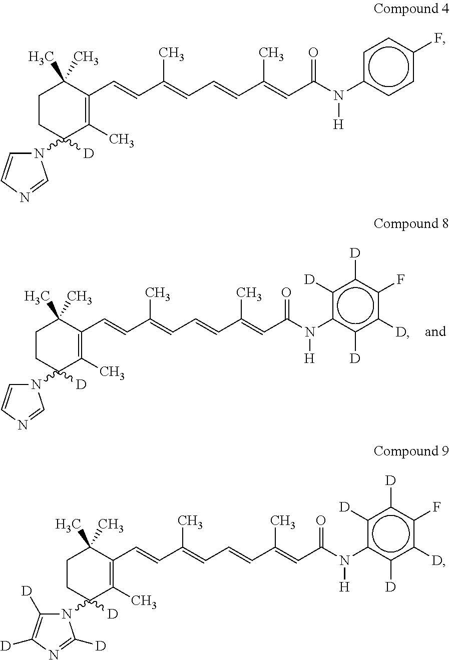

or a pharmaceutically acceptable salt thereof.

The present disclosure also provides a pharmaceutical composition. The pharmaceutical composition may comprise the compound described above or a pharmaceutically acceptable salt thereof, and a pharmaceutically acceptable carrier. In some embodiments, the pharmaceutical composition may be formulated for oral administration.

The present disclosure also provides a method of treating a disease. The method may comprise administering a therapeutically effective amount of the compound described above or a pharmaceutically acceptable salt thereof to a subject in need thereof. In some embodiments, the disease may be one of breast cancer, prostate cancer, bladder, cancer, pancreatic cancer, hepatocellular carcinoma, benign prostatic hyperplasia, Kennedy's disease, and hematologic cancers. In some embodiments, the compound is administered orally.

The present disclosure also provides a method of preparing a compound of formula (A)

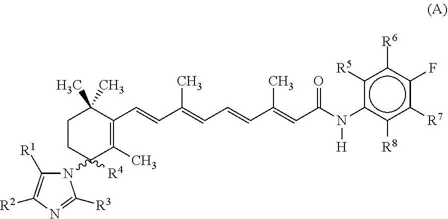

or a salt thereof. The method may comprise converting a compound of formula (B):

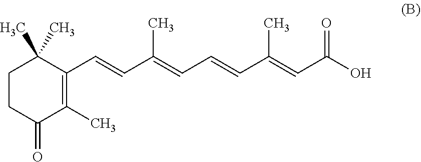

or a salt thereof, into the compound of formula (A) or the salt thereof, wherein each of R¹ to R⁸ is independent H or D, and at least one of R¹ to R⁸ is D.

In some embodiments, the converting of the compound of formula (B) may comprise converting the compound of formula (B) into a compound of formula (C) or a salt thereof or a compound of formula (D) or a salt thereof:

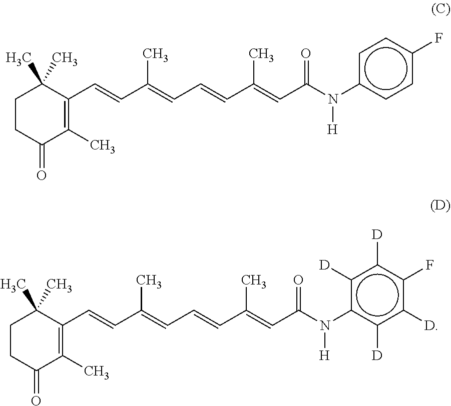

In some embodiments, the method may further comprise converting the compound of formula (C) or the salt thereof into a compound of formula (E) or a salt thereof or a compound of formula (F) or a salt thereof:

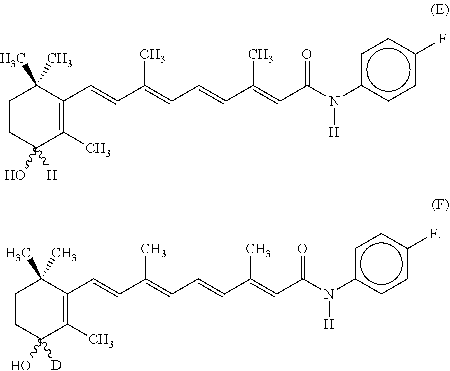

In some embodiments, the method may further comprise converting the compound of formula (D) or a salt thereof into a compound of formula (G) or a salt thereof or a compound of formula (H) or a salt thereof:

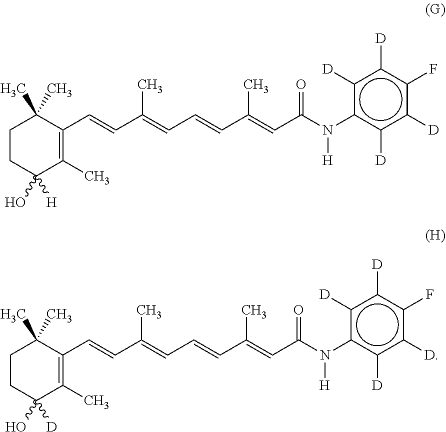

The present disclosure also provides a method of preparing a compound of formula (I)

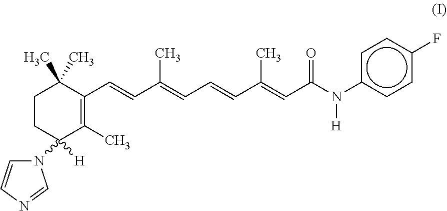

or a salt thereof. The method may comprise converting a compound of formula (B) into the compound of formula (I):

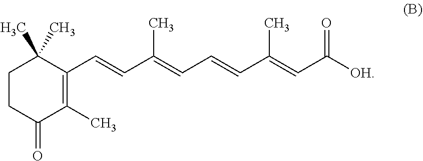

In some embodiments, the converting of the compound of formula (B) may comprise converting the compound of formula (B) into a compound of formula (C):

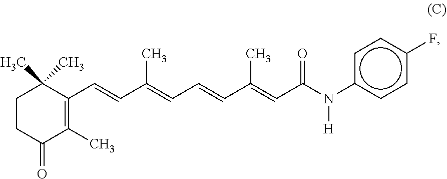

converting the compound of formula (C) into a compound of formula (E):

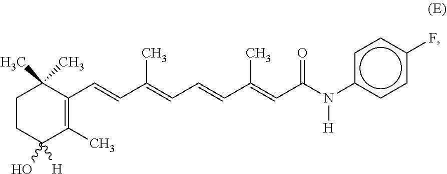

and then converting the compound of formula (E) into the compound of formula (I).

### BRIEF DESCRIPTIONS OF DRAWINGS

The subject matter that is regarded as the invention is particularly pointed out and distinctly claimed in the claims at the conclusion of the specification. The objects, features, and advantages of the present disclosure are apparent from the following detailed description taken in conjunction with the accompanying drawings in which:

FIG. 1 shows the chemical structures of all-trans-retinoic acid (ATRA) (Compound 1), and the developmental candidate, (±)-4-Imidazolyl-4(H)-(4′-fluoro(phenyl))-(E)-retinamide (VNLG-152R, Compound 2).

FIG. 2 shows the predicted metabolic sites on VNLG-152R (Compound 2). The atoms of VNLG-152R with the highest potential for oxidation by Cyp3A4, Cyp2D6 and Cyp2C9 as those marked with circles (◯), and other predicted metabolic sites are marked with a square (□) and a triangle (Δ), representing amide bond cleavage and aromatic ring hydroxylation to form phenols, respectively.

FIG. 3 shows the chemical structures, molecular formulas (MF), and exact masses (EM) of VNLG-152R (Compound 3) and its deuterated analogs (Compounds 3-9).

FIG. 4 shows the synthesis of VNLG-152R from ATRA according to Scheme 1 of the present disclosure.

FIG. 5 shows the synthesis of VNLG-152R from 4-Oxo-ATRA (Compound 15) according to Scheme 2 of the present disclosure.

FIG. 6 shows the synthesis of deuterated retinoidal compounds, Compounds 3-5 of the present disclosure according to Scheme 3 of the present disclosure.

FIG. 7 shows the synthesis of deuterated retinoidal compounds, Compounds 6-9 of the present disclosure according to Scheme 4 of the present disclosure.

FIGS. 8A-8B show a head-to-head comparison of the in vitro antiproliferative effects of non-deuterated VNLG-152R to its deuterated analogs, Compounds 3-9 of the present disclosure, against MDA-MB-231 (FIG. 8A) and MDA-MB-468 (FIG. 8B) human triple negative breast cancer cells.

FIGS. 9A-9B show a head-to-head comparison of the in vitro antiproliferative effects of non-deuterated VNLG-152R to its deuterated analogs, Compounds 3-9 of the present disclosure, and to paclitaxel (PTX), against MDA-MB-231 (FIG. 9A) and MDA-MB-468 (FIG. 9B) human triple negative breast cancer cells.

FIG. 10 shows a schematic of Mnk1/2-eIF4E signaling and the point of action of the disclosed Mnk1/2 degraders.

FIGS. 11A-11B show the effects of VNLG-152R and its deuterated analogs, Compounds 3-9 of the present disclosure, on the in vitro expression of Mnk1, peIF4E, eIF4E, BCL2, BAX, and cyclin D1 in MDA-MB-231 (FIG. 11A) and MDA-MB-468 (FIG. 11B) human triple negative breast cancer cells.

FIGS. 12A-12C show the plasma pharmacokinetics profiles of single individual or cassette PO administration of VNLG-152R and Compounds 3-9 of the present disclosure to CD-1 mice. Data are the mean (±SD) concentrations from three CD-1 mice. FIGS. 12A and 12B represent cassette dosing, and FIG. 12C represents individual dosing.

FIGS. 13A-13D show the anti-tumor activity of VNLG-152R, Compound 4, Compound 8, and Compound 9 in xenograft mouse models. FIG. 13A shows tumor size after 16 days of treatment (PO, 20 mg/kg, 5 days/week) in the MDA-MB-231 model. FIG. 13B shows the percentage change in tumor volumes relative to the volume at the end of experiment for MDA-MB-231 tumor xenograft. FIG. 13C shows tumor size after 16 days of treatment (PO, 20 mg/kg, 5 days/week) in the MDA-MB-468 model. FIG. 13D shows the percentage change in tumor volumes relative to the volume at the end of experiment for MDA-MB-468 tumor xenograft. In FIGS. 13B and 13D, *=p<0.0001.

FIGS. 14A and 14B show the in vitro effects of VNLG-152R on synoviolin 1 (SYVN1) and other targets in MDA-MB-231 cells. FIG. 14A shows that 20 μm VNLG-152R induces SYVN1. FIG. 14B shows that VNLG-152R induces SYVN1 in a dose-dependent manner, and suppresses levels of Mnk1 and downstream targets.

### DETAILED DESCRIPTION

### Cancers

Breast cancer is the second most common cancer among women in the United States, after certain types of skin cancer (Breast Cancer Statistics, Division of Cancer Prevention and Control, Centers for Disease Control and Prevention, available at https://www.cdc.gov/cancer/breast/statistics/index.htm (last viewed Feb. 15, 2022)). Although early diagnosis and new treatment options are effective for the management of primary breast cancer, treatment of advanced breast cancer, specifically metastatic disease, is still a challenge.¹ Triple-negative breast cancers (TNBCs) lack the expression of estrogen receptor (ERα), progesterone receptor (PgR), and human epidermal growth factor receptor 2 (HER2). TNBCs are also the major cause of breast cancer mortality due to aggressive invasive and metastatic potential, lack of suitable molecular treatment targets, and resistance to conventional chemotherapeutic agents.² Hence, there is an urgent need to identify and develop new therapeutic drugs that are effective against TNBCs and metastatic breast cancers (MBCs), especially those that can offer higher survival rates, fewer side effects, and a better quality of life for patients than the currently available therapies.

Meanwhile, prostate cancer is one of the most prevalent malignancies among men worldwide.³ Androgen ablation therapy is typically used to treat advanced and metastatic prostate cancers. However, in most cases, the prostate cancer ultimately becomes unresponsive to androgen ablation and develops into castration-resistant prostate cancer (CRPC), and then progresses rapidly. Therapy for CRPC remains limited and very few drugs have given modest survival in patients with metastatic CRPC (mCRPC).⁴ Thus, as is the case with breast cancers like TNBCs and MBCs, there is an urgency for new effective therapeutic agents for prostate cancers like mCRPC that have potential anti-tumor activity and low toxicity.

Eukaryotic translation initiation factor 4E (eIF4E), a component of the “eIF4F cap binding complex”, plays crucial roles in mRNA discrimination and driving the development and progression of various cancers including breast and prostate cancers. eIF4G-associated eIF4E binds to m7G cap at the 5′-end of eukaryotic mRNAs, initiating translation of “weak mRNAs” encoding malignancy-related proteins.^{5,6} The oncogenic potential of eIF4E is dependent on serine 209 phosphorylation by Mnk1/2, and significantly, eIF4E(S209) phosphorylation by Mnk1/2 is important for tumorigenicity, but not for normal mammalian growth.⁷⁻⁹ eIF4E knockdown is shown to decrease breast cancer cell proliferation in rapamycin-sensitive and rapamycin-insensitive breast cancer cell lines.¹⁰ eIF4E depletion has also been shown to enhance the anti-proliferative and pro-apoptotic effects of chemotherapeutic drugs in breast cancer cells.¹¹ It has been demonstrated that treatment of human breast cancer cells with Mnk1 inhibitors reduced colony formation, proliferation, and survival.¹² The impact of protein translation has also been clearly demonstrated in the clinical setting. For example, a recent prospective study found that >60% of TNBC patients have tumors with high levels of eIF4E, with the conclusion that TNBC patients with high eIF4E overexpression are more likely to recur and die from cancer recurrence and that high eIF4E seems to be a significant prognostic marker in TNBC patients.¹³ eIF4E is reported to be essential for breast cancer progression, angiogenesis,¹⁴ and metastasis.¹⁵ eIF4E elevation of 7-fold or more is a strong prognostic indicator of breast cancer relapse and death in both retrospective and prospective clinical studies.¹⁶⁻¹⁸ In addition, recent reports highlight the impact of inhibiting Mnk-eIF4E signaling in experiment and clinical settings of TNBC.¹⁹⁻²¹

Epithelial to mesenchymal transition (EMT) is a highly conserved process that allows polarized, immobile epithelial cells to transdifferentiate to those with motile mesenchymal phenotypes.^{22,23} EMT produces cancer cells that are invasive, migratory, and exhibit stem cell characteristics that have metastatic potential.²⁴ EMT has been shown to play a pivotal role in the development of mCRPC.^{25,26} Full-length androgen receptor (f-AR) signaling and activation of the Mnk1/2-eIF4E pathway are known to contribute to metastasis by facilitating EMT and promoting signaling interactions.²⁷⁻²⁹ f-AR induces EMT through activation of the Snail transcription factor or via repression of E-cadherin.²⁷ Activation of Snail increases the expression of mesenchymal markers and proteins associated with invasion. In contrast to Snail's activation, transcriptional repression of E-cadherin, a key mediator of intracellular adhesions at adherens junctions, results in collapse of cell-cell communication and onset of EMT.^{30,31}

Activation of eIF4E is recognized to stimulate EMT via promoting the expression of pro-metastatic factors such as Snail and matrix metalloproteinases (MMPs).²⁹ MMPs cleave several component proteins of the extracellular matrix (ECM) to promote tissue invasion and metastasis.^{32,33} It has been demonstrated that besides endorsing tumor development, activation of eIF4E promotes metastatic progression via translation of several EMT-associated mRNAs such as those of Snail and MMP-3.²⁹

There has been increasing interest in the development of agents that inhibit or disrupt oncogenic eIF4F translation complex (and consequently, eIF4E) as strategy to obtain effective anticancer therapeutics against a variety of solid tumors and hematologic cancers.³⁴⁻⁴⁴ Among the various strategies of disrupting the eIF4F complex, inhibition of Mnk1/2 to prevent phosphorylation of eIF4E is generating keen interest. The finding that Mnk1 and Mnk2 regulate mammalian target of rapamycin complex 1 (mTORC1) signaling and associates with mTORC1 directly suggest that Mnk1/2 inhibitors and degraders can also inhibit mTORC1/4E-BP1/p70SK6 signaling.^{45,46} Thus, pharmacological inhibition of Mnk1/2, and, therefore, inhibition of both Mnk-eIF4E and mTORC1 pathways, might serve as a potential effective therapeutic approach for treating patients with advanced breast cancer including TNBCs and MBCs and possibly other malignancies with dysregulated Mnk-eIF4E/m-TORC1 signaling.

### Retinoidal Compounds

Retinoic acid metabolism blocking agents (RAMBA), a family of compounds that inhibit the P450 enzyme(s) responsible for the metabolism of all-trans-retinoic acid (ATRA), have been shown to exert potent anticancer and growth inhibitory effects in vitro in human breast and prostate cancer cells and in vivo in xenograft models.⁴⁷⁻⁵² Retinoidal compounds are a group of natural and synthetic analogues of ATRA and 4-hydroxyphenyl retinamide (4-HPR). Retinoidal compounds comprise a family of polyisoprenoid compounds, and are currently the subject of intense biological interest prompted by the discovery and characterization of retinoid receptor and the realization of these compounds as nonsteroidal small-molecule hormones. Since retinoidal compounds are capable of inhibiting growth, inducing terminal differentiation and apoptosis in cultured cancer cell lines, there is a wide interest in their use in cancer therapy.⁵³ The biological effects of retinoidal compounds result from modulation of gene expression, mediated through two complex types of nuclear receptors, retinoic acid receptors, and retinoid X receptors (RARs and RXRs).⁵⁴ Each type includes 3 distinct subtypes (α, β, and γ) encoded by distinct genes. Each RAR and RXR subtype is expressed in specific patterns in different tissues and is thought to have a specific profile of gene-regulating activity. The nuclear receptors function as dimers. RARs form heterodimers with RXRs. RXRs are more versatile, binding to RARs and other nuclear receptors, including thyroid hormone receptors and vitamin D receptors.

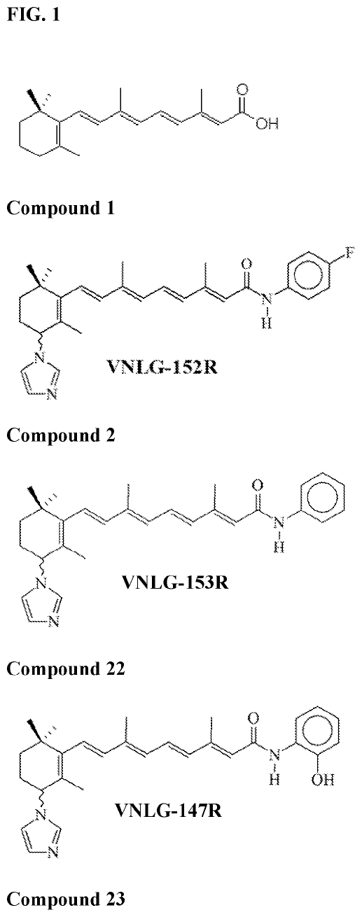

The applicant has designed and synthesized classes of novel retinoidal compounds that exert potent anticancer effects in breast and prostate cancer cells and tumor xenografts by inducing ubiquitin-proteasomal degradation of Mnk1/2 and preventing eIF4E and mTORC1 activation, thereby leading to inhibition of cancer cell growth, apoptosis evasion, cell migration and invasion in vitro and inhibition of tumor xenografts in vivo.^{44,47,48,55,56} One class of the novel retinoidal compounds are based on the ATRA scaffold (Compound 1) (FIG. 1). The ATRA retinoidal compounds are as described in U.S. Pat. Nos. 7,265,143 and 9,156,792, the entire content of each of which is hereby incorporated by reference.

The applicant has demonstrated that the novel ATRA retinoidal compounds simultaneously target both f-AR/androgen receptor splice variant-7 (AR-V7) signaling and MNK-eIF4E translation in several androgen-sensitive cells and CRPC cells in vitro by enhancing f-AR/AR-V7 and MNK degradation through the ubiquitin-proteasome pathway,⁴⁷ and also induce significant down-regulation of f-AR/AR-V7, MNK-1/2 and p-eIF4E to inhibit tumor growth and development mediated by f-AR- and MNK-induced eIF4E activation in xenograft model of CRPC in vivo.⁵⁵ Notable ATRA retinoidal compounds are the lead racemic VNLG-152 (VNLG-152R) (Compound 2, (2E,4E,6E,8E)-9-(3-imidazolyl-2,6,6-trimethylcyclohex-1-enyl)-3,7-dimethyl-N-phenylnona-2,4,6,8-tetraenamide), VNLG-153 (Compound 22, (2E,4E,6E,8E)-9-(3-imidazolyl-2,6,6-trimethylcyclohex-1-enyl)-3,7-dimethyl-N-phenylnona-2,4,6,8-tetraenamide), and VNLG-147 (Compound 23, (2E,4E,6E,8E)-N-(2-hydroxyphenyl)-9-(3-imidazolyl-2,6,6-trimethylcyclohex-1-enyl)-3,7-dimethylnona-2,4,6,8-tetraenamide) (FIG. 1). The applicant has shown that the racemic VNLG-152, VNLG-153, and VNLG-147 exhibit potent anti-proliferative and anti-metastatic activities against androgen-sensitive and castration-resistant human prostate cancer cells in vitro.⁴⁷ The applicant has also shown that VNLG-152R (Compound 2) exerts anti-cancer and anti-metastatic activities in in vivo models of breast and prostate cancers and demonstrated the compound's pharmacokinetics, including oral bioavailability in mice.⁵⁵⁻⁵⁶

### Deuterated Retinoidal Compounds

The utility of deuterium (²H, D) substitution for hydrogen (¹H, H) to alter pharmacokinetics and metabolism in small molecule pharmaceuticals is a valuable approach in medicinal chemistry and drug discovery and development.⁵⁷⁻⁵⁹ Indeed, the first and classical example is deuterobennazine that was approved by the U.S. Food and Drug Administration (FDA) in 2017 for the treatment of chorea with Huntington's disease,^{57,60-62} and >20 deuterated agents are currently in clinical development, including deuterated C₂₀-D₃-vitamin A (ALK-001), which is currently being investigated in a Phase 3 study for treating geographic atrophy (GA) secondary to age-related macular degeneration (AMD).^{57,60,69}

The present disclosure provides novel deuterated analogs of the applicant's novel ATRA retinoidal compounds. The applicant hypothesizes that deuterium substitution for hydrogen at potential metabolic hot spots on retinoidal compounds including VNLG-152R (Compound 2) can slow down in vivo metabolism of the compounds to enable improved pharmacokinetic properties. Indeed, modest half-life improvements (prolonging the residence time) for short half-life compounds can dramatically lower the efficacious dose.^{56,64}

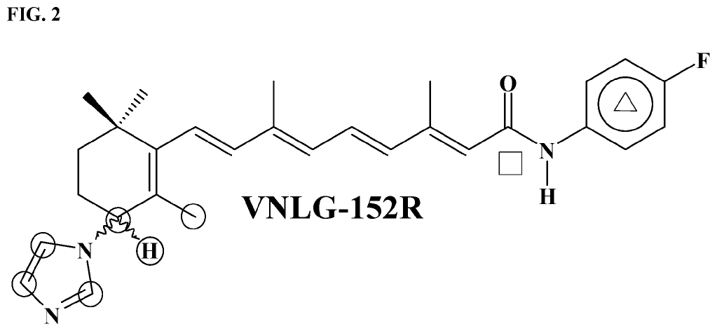

To design the possible deuterated analogs of VNLG-152R (Compound 2), the applicant has identified atoms of VNLG-152R (Compound 2) with the highest potential for oxidation by Cyp3A4, Cyp2D6 and Cyp2C9 as those marked with circles (◯). Other predicted metabolic sites are marked with a square (□) and a triangle (Δ), representing amide bond cleavage and aromatic ring hydroxylation to form phenols, respectively. FIG. 2 maps the predicted metabolic sites onto VNLG-152R.

The applicant has discovered that the amide hydrogen atom was readily exchangeable, and based on this discovery, has focused on the replacement of the C4-H, the three hydrogens of C4-imidazole and the four hydrogens of amide benzyl fluoride. The possible exchange of hydrogen(s) with deuterium(s) at the three regions in VNLG-152R (Compound 2) translates to 7 (factorial of 3 (3!, i.e., 3×2×1=6)+1) deuterated analogs of VNLG-152R (Compound 2). The present disclosure thus provides a deuterated retinoid of Formula A, or a pharmaceutically acceptable salt thereof:

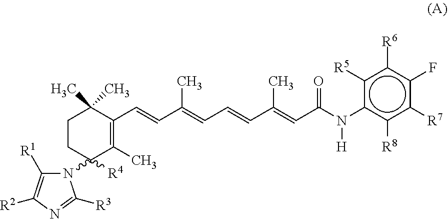

In Formula A, each of R¹ to R⁸ is independent H or D, and at least one of R¹ to R⁸ is D. In some embodiments, R⁴ is D. In some embodiments, R⁴ and at least one of R⁶ to R⁸ is D. In some embodiments, the deuterated retinoid is at least one selected from the following, or a pharmaceutically acceptable salt of at least one selected from the following:

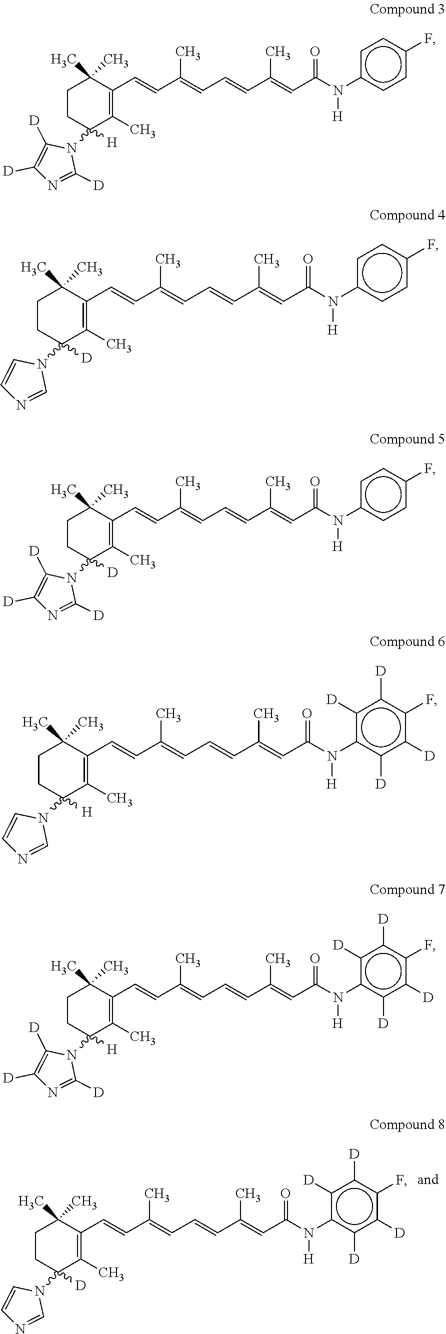

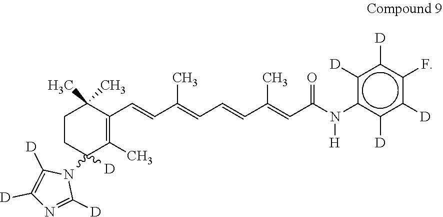

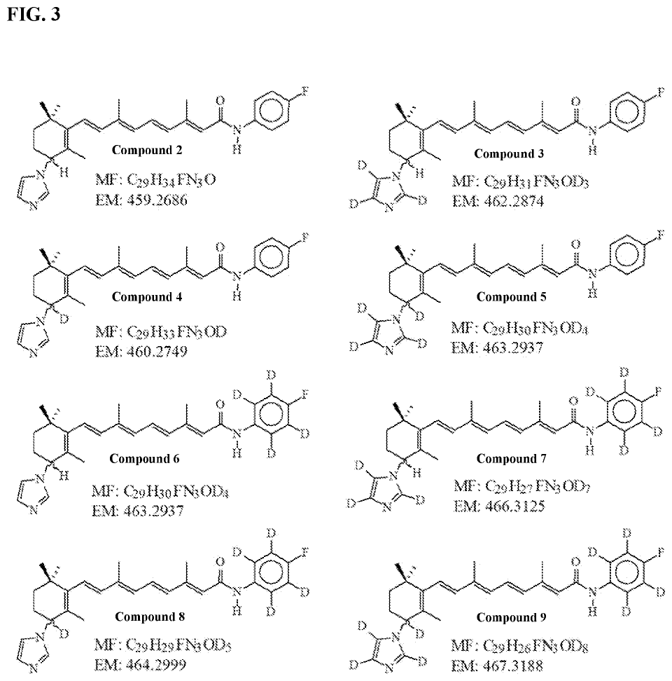

The structures and exact masses of the deuterated Compounds 3-9 are shown in FIG. 3.

### Synthesis and Preparation of VNLG-152R

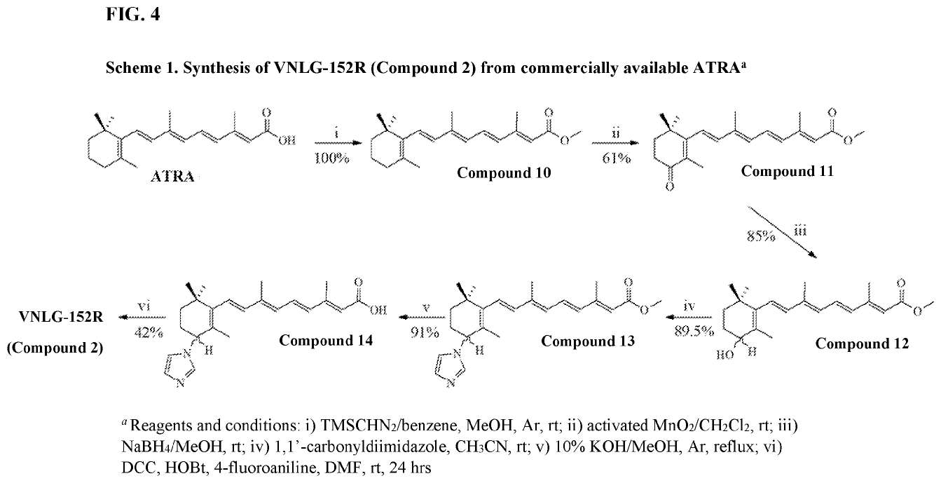

VNLG-152R (Compound 2) was previously synthesized in one step from the applicant's early lead compound, VN/14-1 following treatment with commercially available 4-fluoroaniline in the presences of 1-hydroxy benzotriazole (HOBT) and dicyclohexylcarbodiimide (DCC) in dimethyl formamide at room temperature. For the sake of clarity, the synthesis of VN/14-1 that involved five steps from the commercially available ATRA and the synthesis of VNLG-152R (Compound 2), including the yields of each step is presented in Scheme 1 in FIG. 4. Thus, the overall yield for the synthesis of VNLG-152R (Compound 2) from ATRA is 18%. The relatively low yields of step two that involves the allylic oxidation of Compound 10 with manganese dioxide and the DCC amide formation step six have the most significant impact on the low overall yield of VNLG-152R (Compound 2).

Because of the further ongoing development of VNLG-152R (Compound 2) towards clinical trials in humans and because of the need to produce new analogs of VNLG-152R (Compound 2) with enhanced pharmacokinetics and as a strategy to generate entirely new chemical entities, the applicant has designed a remarkably improved procedure for the synthesis of VNLG-152R (Compound 2).

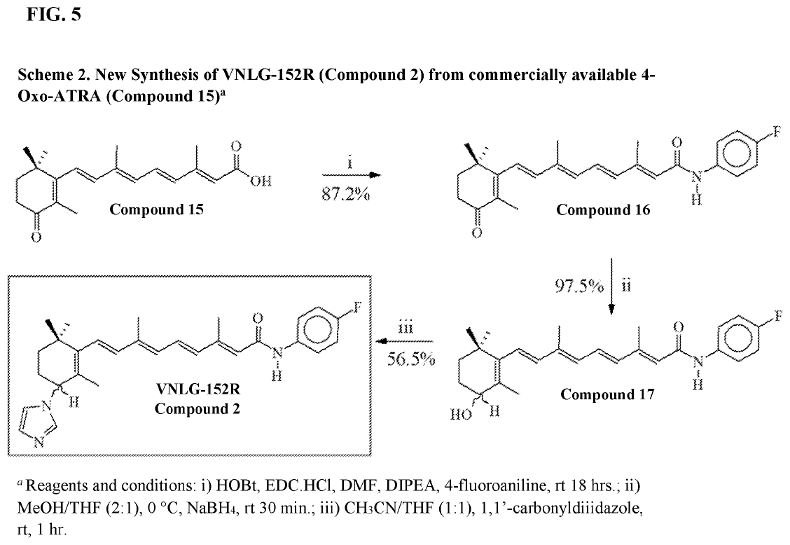

Recently, 4-oxo-ATRA (Compound 15) became commercially available. Drawing from experience with the previous synthesis of VNLG-152R (Compound 2) from ATRA, the applicant envisions that using 4-oxo-ATRA (Compound 15) as starting material would eliminate the low yielding allylic oxidation step in Scheme 1 (FIG. 4). The novel synthesis of VNLG-152R (Compound 2) is outlined in Scheme 2 in FIG. 5.

The 4-oxo-retinamide (Compound 16) is synthesized by coupling 4-oxo-ATRA (Compound 15) with 4-fluoroaniline by the active ester method using 1-ethyl-3-[3-dimethylaminopropyl]carbodiimide hydrochloride (EDC.HCl) and 1-hydroxbenzotriazole (HOBt). This reagent offers advantages over the previously used DCC method due to ease in work-up without requiring column chromatographic purification to give pure 4-oxo-retinamide intermediate (Compound 16) in excellent yield (87%). Reduction of Compound 16 with sodium borohydride (NaBH₄) using a solvent mixture of methanol/tetrahydrofuran at 2:1 ratio provides the key intermediate (±)-4-hydroxy retinamide (Compound 17) in near quantitative (97%) yield. The production of Compound 17 does not require column chromatographic purification. Finally, treatment of Compound 17 with carbonyldiimidazole (CDI) using a solvent mixture of acetonitrile/tetrahydrofuran at 1:1 ratio at room temperature provides the desired VNLG-152R (Compound 2) in 56.5% yield after chromatographic purification. In this final step, the applicant has developed an efficient chromatographic purification method which removed colored/unidentified impurities which has very close retention times with VNLG-152R (Compound 2). The novel Scheme 2 synthesizes VNLG-152R (Compound 2) at an overall yield of 48%, which substantially improves on the 18% overall yield that is possible with Scheme 1.

### Synthesis and Preparation of Deuterated Retinoidal Compounds

The present disclosure also provides methods for synthesizing deuterated analogs of the ATRA. The deuterated compounds disclosed in the present disclosure may be synthesized by the routes described below and in the accompanying figures. Materials used in the described routes are either commercially available or prepared by synthetic methods generally known in the art. These schemes are not limited to the compounds listed or by any particular substituents employed for illustrative purposes.

The deuterated Compounds 3-9 are synthesized through three-step synthetic routes like Scheme 2 for the parent VNLG-152R (Compound 2).

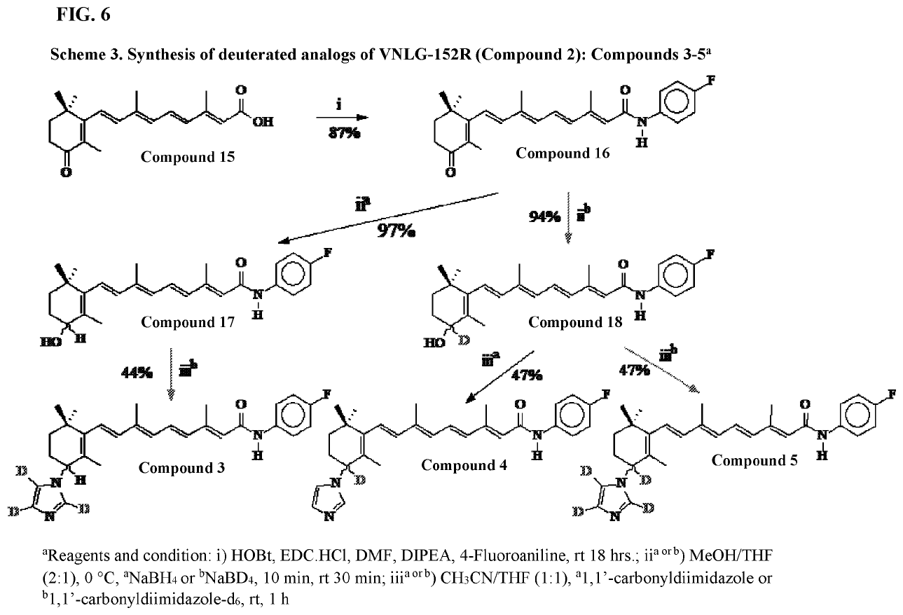

In one aspect, the present disclosure provides a method for synthesizing Compounds 3-5 according to the route shown in Scheme 3 in FIG. 6. The key intermediate for the synthesis of the deuterated analogs Compounds 3-5 is the 4-oxo-retinamide (Compound 8). As shown in Scheme 3 in FIG. 6, the 4-oxo-retinamide (Compound 8) is treated with either NaBH₄ or NaBD₄ to give the corresponding non-deuterated 4-hydroxy Compound 17 and the 4H deuterated 4-hydroxy Compound 18. Treatment of Compound 17 with 1,1′-carbonyldiimidazole-d₆, at room temperature provides the desired (±) 4-(Imidazolyl-2″3″5″-D₃)-4-(H)-(4′-fluoro(phenyl))-(E)-retinamide (Compound 3). Treatment of Compound 18 with either 1,1′-carbonyldiimidazole or 1,1′-carbonyldiimidazole-d₆ provides the desired (±) 4-(Imidazolyl)-4-(D)-(4′-fluoro(phenyl))-(E)-retinamide (Compound 4) and (±) 4-(Imidazolyl-2″3″5″-D₃)-4-(D)-(4′-fluoro(phenyl))-(E)-retinamide (Compound 5), respectively.

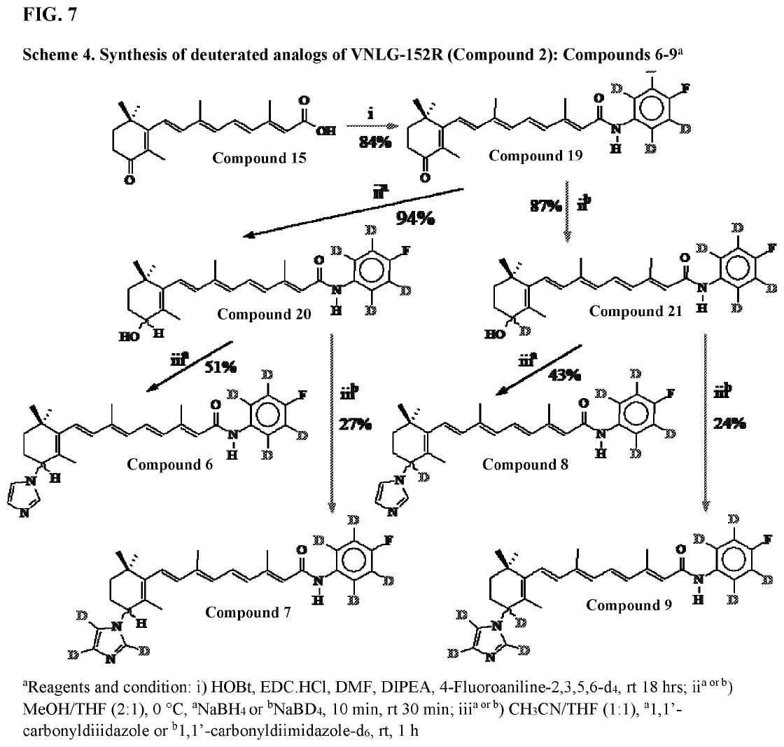

In another aspect, the present disclosure provides a method for synthesizing Compounds 6-9 according to the route shown in Scheme 4 in FIG. 7. The deuterated analogs Compounds 6-9 are synthesized in a similar fashion as Compounds 3-5 in Scheme 3, but as shown in Scheme 4 in FIG. 7, instead of 4-oxo-retinamide (Compound 8), the key intermediate in the synthesis of Compounds 6-9 is 4-oxo-(4′-fluoro(phenyl-2′3′5′6′-D₄)-(E)-retinamide (Compound 19). Notably, the yields for the final steps for all the deuterated analogs are modest, which the applicant hypothesizes is attributable to the poor solubility of the deuterated reagents.

The applicant has developed a new and improved procedure to synthesize the developmental VNLG-152R (Compound 2). The improved synthesis and yield of VNLG-152R enables the synthesis of novel deuterated Compounds 3-9, which are deuterated analogous of VNLG-152R. The novel deuterated analogs of the present disclosure prove to be potent Mnk1 degrader with consequent depletion of peIF4E and other oncogenic proteins. Importantly, the in vivo pharmacokinetics for both the non-deuterated VNLG-152R (Compound 2) and the deuterated analogs (Compounds 3-9) show that the pharmacokinetic parameters of the deuterated analogs, and in particular, Compounds 4, 8, and 9, are superior to the non-deuterated VNLG-152R in terms of Cₘₐₓ, T_{1/2}, AUC and MRT. The therapeutic potential of the deuterated analogs of the present disclosure is promising. Significantly, because the deuteration strategy described in the present disclosure has generated entirely novel chemical entities, the new composition of matter will undoubtedly support further development of these promising deuterated compounds.

### Pharmaceutical Compositions

The deuterated retinoid or its salt of the present disclosure can be used to prepare a pharmaceutical composition effective for treating conditions associated with Mnk1/2 signaling and/or androgen signaling, including, for example, breast cancer, prostate cancer, bladder, cancer, pancreatic cancer, hepatocellular carcinoma, benign prostatic hyperplasia (BPH), and Kennedy's disease (spinal and bulbar muscular atrophy), and hematologic cancers. In some embodiments, the deuterated compound or its salt of the present disclosure is used to prepare pharmaceutical compositions effective for treating triple negative breast cancer.

The present disclosure provides a pharmaceutical composition comprising a therapeutically effective amount of at least one deuterated retinoidal compound described above. The deuterated retinoidal compound (also referred to in this disclosure as “active compounds”) or its salt of the present disclosure can be incorporated into pharmaceutical compositions suitable for administration. Such compositions typically comprise the deuterated retinoidal compound or its salt and a pharmaceutically acceptable carrier.

The pharmaceutical composition may comprise a deuterated retinoidal compound of Formula A, or a pharmaceutically acceptable salt thereof:

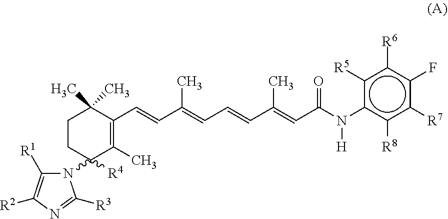

In Formula A, each of R¹ to R⁸ is independent H or D, and at least one of R¹ to R⁸ is D. In some embodiments, R⁴ is D. In some embodiments, R⁴ and at least one of R⁶ to R⁸ is D.

In some embodiments, the pharmaceutical composition comprises at least one of Compounds 3-9 or a salt thereof. In some embodiments, the pharmaceutical composition comprises at least one of Compounds 4, 8, and 9 or a salt thereof. In some embodiments, the pharmaceutical composition comprises a mixture of at least one ATRA retinoidal compound, or a salt there of, and at least one of Compounds 3-9 or a salt thereof.

As used in this application, “pharmaceutically acceptable carrier” refers to those components in the particular dosage form employed, which are considered inert and are typically employed in the pharmaceutical arts to formulate a dosage form containing a particular active compound. The pharmaceutically acceptable carrier includes any and all solvents, dispersion media, coatings, antibacterial and antifungal agents, isotonic and absorption delaying agents, and the like, that is compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A deuterated retinoidal compound of the present disclosure can be used as a monotherapy or in combination with other therapeutic agents. The deuterated retinoidal compound or its salt can be used in combination with other cancer treatments and drugs. For example, the deuterated retinoidal compound or its salt of this disclosure can be used as a part of or in combination with known cancer treatments such as hormone therapy, chemotherapy, radiation therapy, immunotherapy, and/or surgery. The deuterated retinoidal compound or its salt of this disclosure can also be used in combination with one or more known and available drugs or other compounds. The therapeutic effectiveness of one of the deuterated retinoidal compounds or their salts described in this disclosure may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering one of the deuterated retinoidal compounds or their salts of the present disclosure with another therapeutic agent (which also includes a therapeutic regimen) that also has therapeutic benefit. In any case, regardless of the disease or condition being treated, the overall benefit experienced by the patient may be synergistic of the multiple therapeutic agents or the patient may experience a synergistic benefit. Where the deuterated retinoidal compounds or their salts of the present disclosure are administered in conjunction with other therapies, dosages of the co-administered therapeutic agents will of course vary depending on the type of co-drug employed, on the specific drug employed, on the disease or condition being treated and so forth. In addition, when co-administered with one or more biologically active agents, the deuterated retinoidal compounds or their salts of the present disclosure may be administered either simultaneously with the biologically active agent(s), or sequentially. If administered simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If administered sequentially, the attending physician will decide on the appropriate regimen. In addition, the combination methods, compositions and formulations are not to be limited to the use of only two agents. Multiple therapeutic combinations are envisioned.

As used in this application, “therapeutically effective amount” of a deuterated retinoidal compound or its salt is the amount that effectively achieves the desired therapeutic result in the subject. Such amounts may be initially determined by knowledge in the art, by conducting in vitro tests, by conducting metabolic studies in healthy experimental animals, and/or by conducting clinical trials. Naturally, the dosages of the various deuterated retinoidal compounds or their salts of the present disclosure will vary somewhat depending upon the host treated, the particular mode of administration, among other factors. Those skilled in the art can determine the optimal dosing of the deuterated retinoidal compound or its salt of the present disclosure selected based on clinical experience and the treatment indication.

The pharmaceutical composition according to the present disclosure is configured to facilitate administration of a deuterated retinoidal compound or its salt to a subject. A deuterated retinoidal compound or its salt of the present disclosure, or a pharmaceutical composition containing the deuterated retinoidal compound or its salt, can be administered according to any suitable method or route, including, but not limited to, intranasal, intramuscular, intratracheal, subcutaneous, intradermal, transdermal, sublingual, topical application, intravenous, ocular (e.g., topically to the eye, intravitreal, etc.), rectal, nasal, oral, topical administration, and other enteral and parenteral routes of administration.

In some embodiments, the pharmaceutical composition is formulated for an oral administration. In some embodiments, the pharmaceutical composition may comprise an amount of the deuterated retinoidal compound or its salt sufficient to administer from 0.01 to 100 mg/kg of body weight per day, such as 0.01 to 35 mg/kg of body weight per day, 0.05 to 20 mg/kg of body weight per day, or 5 to 20 mg/kg of body weight per day, of the deuterated retinoidal compound or its salt.

Compositions suitable for oral administration can contain (a) liquid solutions, such as an effective amount of one or more compound or salt of this disclosure dissolved in a diluent, such as water or saline, (b) capsules, sachets or tablets, each containing a predetermined amount of the active ingredient, as solids or granules, (c) suspensions in an appropriate liquid, and (d) suitable emulsions. For oral administration, a deuterated retinoid or its salt of the present disclosure can be formulated, for example, by combining the active compounds with pharmaceutically acceptable carriers or excipients known in the art. Such carriers enable a retinamide of the present disclosure to be formulated as tablets, powders, pills, dragees, capsules, liquids, gels, syrups, elixirs, slurries, suspensions and the like, for oral ingestion by a patient to be treated.

In some embodiments, the pharmaceutical composition is formulated for parenteral administration. Compositions suitable for parenteral administration include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried condition requiring only the addition of the sterile liquid carrier, for example, water, for injections, immediately prior to use. Extemporaneous injection solutions and suspensions can be prepared from sterile powders, granules, and tablets of the kind previously described.

Delivery of a deuterated retinoidal compound or its salt may be enhanced by promoting a more pharmacologically effective amount of the compound reaching a site of action. The delivery may also be enhanced by promoting a more effective delivery of the compound across a cell membrane or within the cell and across the intra-cellular space.

Non-limiting examples of suitable carriers or excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include lubricating agents such as talc, magnesium stearate, and mineral oil; wetting agents; emulsifying and suspending agents; preserving agents such as methyl- and propyl-hydroxybenzoates, sweetening agents; and flavoring agents.

A deuterated retinoidal compound or its salt of the present disclosure may be applied to exert a local or a systemic effect, or both. For example, where systemic delivery is desired, administration may involve invasive or systemically absorbed topical or mucosal administration of pharmaceutical preparation. A pharmaceutical composition containing a deuterated retinoidal compound or its salt of the present disclosure may be administered in a targeted drug delivery system. In addition, a pharmaceutical composition containing a deuterated retinoidal compound or its salt of the present disclosure may be formulated according to suitable procedures known in the art to be a rapid release formulation, an extended release formulation, or an intermediate release formulation.

The present disclosure provides methods for preparing pharmaceutical compositions containing a deuterated retinoidal compound or its salt of the present disclosure. Such compositions can further include additional active agents. Thus, the present disclosure further provides methods for preparing a pharmaceutical composition by formulating a pharmaceutically acceptable carrier with a deuterated retinoidal compound or its salt of the invention and one or more additional active compounds.

### Treatment Methods

The present disclosure also provides methods for treatment of various conditions, especially conditions associated with Mnk1/2 and/or androgen signaling. In some embodiments, the methods involve administering to a subject in need thereof (for example, a human or a non-human mammal) a therapeutically effective amount of a deuterated retinoidal compound or its salt of the present disclosure, or a pharmaceutical composition comprising a therapeutically effective amount of the deuterated retinoidal compound or its salt. In some embodiments, the deuterated retinoidal compound or its salt of the present disclosure is administered to treat at least one selected from the group consisting of breast cancer, prostate cancer, bladder, cancer, pancreatic cancer, hepatocellular carcinoma, benign prostatic hyperplasia (BPH), and Kennedy's disease (spinal and bulbar muscular atrophy), and hematologic cancers. In some embodiments, the condition is triple negative breast cancer. The deuterated retinoidal compound may comprise at least one of Compounds 3-9 or a salt thereof. In some embodiments, the deuterated retinoid may comprise at least one of Compounds 4, 8, and 9 or a salt thereof.

In some embodiments, the deuterated retinoidal compound or its salt is administered orally or parenterally.

The “therapeutically effective amount” is as defined above. The dose administered to an animal, particularly a human, in the context of the present invention should be sufficient to affect a therapeutic response in the animal or human over a reasonable time frame. The specific dose level and frequency of dosage may vary, depending upon a variety of factors, including the activity of the specific active compound, its metabolic stability and length of action, rate of excretion, mode and time of administration, the age, body weight, health condition, gender, diet, etc., of the subject, and the severity of, for example, the breast cancer. Any effective amount of the compound can be administered, e.g., from about 1 mg to about 500 mg per day, about 50 mg to about 150 mg per day, etc. In some embodiments, the deuterated retinoidal compound or its salt may be administered at 0.01 to 100 mg/kg of body weight per day, such as 0.01 to 35 mg/kg of body weight per day, 0.05 to 20 mg/kg of body weight per day, or 5 to 20 mg/kg of body weight per day, of the deuterated retinoidal compound or its salt. The deuterated retinoidal compound or salt of this disclosure can be administered in such dosages in any form by any effective route, including, e.g., oral, parenteral, enteral, intraperitoneal, topical, transdermal (e.g., using any standard patch), ophthalmic, nasally, local, non oral, such as aerosol, spray, inhalation, subcutaneous, intravenous, intramuscular, buccal, sublingual, rectal, vaginal, intra arterial, and intrathecal, etc. In some embodiments, the deuterated retinoidal compound or its salt is administered once daily.

A deuterated retinoidal compound or salt of the present disclosure may be used in combination with procedures that may provide additional or synergistic benefit to the patient. A deuterated retinoidal compound or salt of the present disclosure, whether as monotherapy or in combination therapy, may be administered before, during or after the occurrence of the disease or condition to be treated, and the timing of administering the composition containing the deuterated retinoid or salt can vary. Thus, a deuterated retinoidal compound or salt may be administered to a subject during or as soon as possible after the onset of the symptoms. A deuterated retinoidal compound or salt is preferably administered as soon as is practicable after the onset of the disease or condition enumerated in the present disclosure is detected or suspected, and for a length of time necessary for the treatment of the disease. The length of treatment can vary for each subject, and the length can be determined using known criteria. Administration can be acute (for example, of short duration (e.g., single administration, administration for one day to one week)), or chronic (for example, of long duration, (e.g., administration for longer than one week, from about 2 weeks to about one month, from about one month to about 3 months, from about 3 months to about 6 months, from about 6 months to about 1 year, or longer than one year)).

As used in this application, “treat” and all its forms and tenses (including, for example, treat, treating, treated, and treatment) refer to therapeutic treatment and/or prophylactic or preventative treatment. Those in need of treatment include those already with a pathological condition enumerated in the present disclosure as well as those in which the pathological condition is to be prevented. For example, “treat” means alter, apply, effect, improve, care for or deal with medically or surgically, ameliorate, cure, stop and/or prevent an undesired biological (pathogenic) process. Those of ordinary skill in the art are aware that a treatment may or may not cure.

### EXAMPLES

The following examples are offered to illustrate the present invention and to assist one of ordinary skill in the relevant art in making and using the present invention. The examples are not intended to limit the scope of the present invention.

### Example 1

All the materials listed below were purchased and used without further purification. The starting material 4-oxo-ATRA was purchased from Synnovator, Inc., 11 Centrewest Ct, Cary, NC 27513, USA. 1,1′-carbonyldiimidazole-d₆ and 4-fluoroaniline-2,3,5,6-d₄ were purchased from CDN Isotopes Inc., 88 Leacock, Poine-Claire, QC H9R 1H1, Canada. All other reagents and solvents of research grade or spectrophotometric grade in the highest purity were commercially available from Sigma-Aldrich. Silica gel plates (Merck F254) were used for thin layer chromatography (TLC) and were developed with mixtures of ethyl acetate (EtOAc)/Petroleum ether (1:3) or 1% ethanol in EtOAc, unless otherwise specified and were visualized with 254 and 365 nm light. Petroleum ether refers to light petroleum, boiling point (bp) 40-60° C. Column chromatographic purifications were performed using normal phase silica columns on CombiFlash Teledyne ISCO automated chromatography instrument. ¹H and ¹³C NMR spectra were recorded in CDCl₃ or DMSO-d₆ using a Bruker 400 MHz NMR instrument and chemical shifts are reported in ppm on the δ scale relative to tetramethylsilane. ²H NMR spectra were recorded in 0.5% DMSO-d₆ in DMSO using a Bruker Avance III 950 MHz NMR instrument. High resolution mass spectra were obtained on Bruker 12 T APEX-Qe FTICR-MS instrument by positive ion ESI mode by Cosmic Laboratory, Old Dominion University, Norfolk, VA. Melting points (mp) were determined with Fischer Johns melting point apparatus uncorrected. Purities of the compounds were determined by reverse phase on LC system of Waters Acquity UPLC with a photodiode array detector, using Novapac-C18 (3.9×150 mm×4 m) column as stationary phase. For mobile phase Solvent-A contained 20 mM ammonium acetate in Methanol-Water (70:30 v/v), solvent-D is methanol, flow rate 0.5 mL/min and maintained gradient flow (for gradient flow details please see chromatograms in supplemental section). The column maintained at room temperature and injection volume 10.0 μL. The purities of all final compounds were determined to be at least 95% pure by a combination of UPLC, NMR and HRMS.

### General Method A: Synthesis of 4-oxo-retinamides (Compounds 16 and 19) from 4-oxo-ATAR (Compound 15)

In General Method A, a suspension of 4-oxo-ATRA (Compound 15) (1 molar ratio (MR)), 1-hydroxbenzotriazole (2 MR), 1-ethyl-3-[3-dimethylaminopropyl]carbodiimide hydrochloride (EDC.HCl) (2 MR), dimethylformamide (5 mL/g of substrate) is added N,N-diisopropylethylamine (10 MR) and 4-fluoroaniline or 4-fluoroaniline-2,3,5,6-d₄ (1.1 MR). The reaction mixture is sonicated for five minutes to obtain a homogenous solution and then stirred at room temperature for 18 hours. The excess of diisopropylethylamine from the reaction mixture is stripped off under vacuum. To the remaining mixture, about 25 mL of ice/water is added and vigorously stirred until fine solids are obtained, then 25 mL more water is added (10 mL/mL of DMF). The product is filtered at room temperature, washed thoroughly with water, filtered and dried. The resulting orange solid is recrystallized with hot ethanol (2 mL/g of product) and filtered at room temperature to obtain orange needles. This product is in the next step of the synthetic scheme without further purification.

### Example 2

### General Method B: Conversion of 4-Oxo-Retinamides (Compounds 16, 19) to Hydroxy-Retinamides (Compounds 17, 18, 20, and 21)

The reagents, materials, and instrumentations are as described in Example 1. In General Method B, a solution of 4-oxo-retinamide (Compounds 16, 19) (1 MR), methanol (10 mL/g of substrate), and tetrahydrofuran (5 mL/g of substrate) is cooled to ice temperature (˜4° C.). Sodium borohydride or sodium borodueteride (1.2 MR) is added in three portions over fifteen minutes and the reaction mixture is stirred at room temperature for 30 minutes. To the resultant yellow solution (orange to yellow), acetone (2 mL/g of substrate) is added and stirred for five minutes. The reaction mixture is then concentrated under vacuum, treated with ice water, filtered, and dried under vacuum. This product is used in the next step of the synthetic scheme without further purification.

### Example 3

### General Method C: Appending Imidazole at C-4 Position (1, 10-16) to Synthesize VNLG-152R (Compound 2)

The reagents, materials, and instrumentations are as described in Example 1. The following General Method C is used to synthesize VNLG-152R (Compound 2) from the intermediate 4-hydroxy-retinamide (Compounds 17, 18, 20, 21) by appending imidazole at the C-4 position. In General Method C, a solution of 4-hydroxy-retinamide (Compounds 17, 18, 20, 21) (1 MR) in acetonitrile (5 mL/g of substrate), and tetrahydrofuran (5 mL/g of substrate) is added 1,1′-carbonylimidazole or 1,1′-carbonyldiidazole-d₆ (1.5 MR) and stirred for 1.5 hours at room temperature. The initial yellow solution becomes orange. The solvents are evaporated under vacuum, reconstituted with dichloromethane (3 mL/g of substrate), water added (6 ml/g of substrate), and dichloromethane stripped off from mixture under vacuum. This process is repeated twice to solubilize unreacted CDI and imidazole in the aqueous phase. The resulting solids are filtered, or water decanted if the residue is sticky, and the contents are dried under vacuum. The crude product is purified by silica gel column (five-inch) using solvent A (ethyl acetate) for 2 min then solvent B (ethyl acetate/Ethanol/trimethylamine at a ratio of 95:5:0.5) from 0 to 5% over 20 minutes on the CombiFlash automated chromatography instrument. The fractions between 10.5 to 18 minutes are combined and evaporated to obtain yellow solids.

### Example 4

### (±) 4-Imidazolyl-4(H)-(4′-fluoro(phenyl))-(E)-retinamide (VNLG-152R, Compound 2)

Compound 2 (VNLG-125R) was synthesized using General Method C in Example 3. A solution of (±) 4-Hydroxy-4-[H]-(4′-fluoro(phenyl)-(E)-retinamide (Compound 17) (0.94 g, 2.29 mmol) in acetonitrile (5 mL) and tetrahydrofuran (5 mL) was added 1,1′-cabonyldiimidazole (0.56 g 3.44 mmol) and stirred for 1.5 hours at room temperature (initial yellow solution becomes orange). Solvent evaporated under vacuum, reconstituted with dichloromethane (3 mL), water added, and dichloromethane stripped off from mixture under vacuum. This process was repeated twice to solubilize unreacted CDI and imidazole in aqueous phase. The resulting solids were filtered (0.9 g) and purified by silica gel column (five-inch) using solvent A (ethyl acetate) for 2 min then solvent B (ethyl acetate/Ethanol/trimethylamine 95:5:0.5) from 0 to 5% over 20 minutes on the CombiFlash an automated chromatography instrument. The fractions between 10.5 to 18 minutes were combined and evaporated to obtain yellow solids; 0.52 g (49%); mp, sinters at 105° C., melts at 111-112° C.; ¹H NMR (400 MHz, CDCl₃) δ ppm 1.03-1.19 (m, 6H) 1.45-1.59 (m, 2H) 1.59-1.64 (m, 3H) 1.81-1.89 (m, 1H) 2.02 (s, 3H) 2.07-2.16 (m, 1H) 2.31-2.53 (m, 3H) 4.54 (t, 1H) 5.88 (br. s., 1H) 6.06-6.22 (m, 2H) 6.22-6.37 (m, 2H) 6.93 (s, 1H) 6.93-7.06 (m, 3H) 7.09 (s, 1H) 7.52 (s, 1H) 7.55 (br. s., 2H) 8.14 (br. s., 1H); ¹H NMR (400 MHz, DMSO-d₆) δ ppm 1.08 (s, 3H) 1.11 (s, 3H) 1.40-1.59 (m, 5H) 1.73-1.82 (m, 1H) 2.01 (s, 3H) 2.02-2.12 (m, 1H) 2.23-2.41 (m, 3H) 4.72 (t, 1H) 6.02 (s, 1H) 6.28 (m, 2H) 6.31-6.47 (m, 2H) 6.92 (s, 1H) 7.01 (dd, 1H) 7.07-7.20 (m, 3H) 7.62 (s, 1H) 7.66 (dd, 2H) 10.07 (s, 1H). ¹³C NMR (101 MHz, DMSO-d₆) δ ppm 13.0, 13.7, 18.8, 28.1, 28.3, 29.1, 34.7, 35.1, 57.4, 115.5, 115.8, 118.8, 121.0, 121.1, 123.2, 125.8, 126.9, 128.8, 130.2, 131.6, 136.3, 136.3, 137.0, 137.3, 138.3, 139.0, 143.9, 148.8, 157.1, 159.5, 165.0; HRMS calcd 460.2758 (C₂₉H₃₄FN₃O H+) found 460.2760.

### Example 5

### 4-Oxo-(4′-fluoro(phenyl))-(E)-retinamide (Compound 16)

Compound 16 was synthesized using General Method A in Example 1. To a suspension of 4-Oxo-ATRA (Compound 15) (2 g, 6.36 mmol), 1-hydroxybenzotriazole (1.71 g, 12.7 mmol), 1-ethyl-3-[3-dimethylaminopropyl]carbodiimide hydrochloride (EDC.HCl) (2.44 g, 12.7 mmol), dimethylformamide (10 mL) added N,N-diisopropylethylamine (8.22 g, 63.6 mmol) and 4-fluoroaniline (0.78 g, 7.0 mmol) and sonicated for 5 minutes to obtain a homogenous solution. Thereafter, the mixture was stirred at room temperature for 18 hours. The excess if diisopropylethylamine was stripped off under vacuum. To the remaining mixture, about 50 mL of ice/water added and vigorously stirred until fine solids obtained, then, 50 mL more water added. The product filtered at room temperature and washed thoroughly with water, filtered and dried. The resultant orange solid (2.49 g) of 4-Oxo-(4′-fluoro(phenyl)-(E)-retinamide (Compound 16) was recrystallized using hot ethanol (5 mL) to obtain orange needles; 2.26 g (87%); mp, sinters at 166° C., melts at 171° C. ¹H NMR (400 MHz, CDCl₃) δ ppm 1.20 (s, 6H) 1.79-1.92 (m, 5H) 2.04 (s, 3H) 2.43 (s, 3H) 2.52 (t, 2H) 5.87 (s, 1H) 6.26 (d, 1H) 6.30-6.41 (m, 3H) 6.92-7.07 (m, 3H) 7.40 (br. s., 1H) 7.54 (br. s., 2H); ¹H NMR (400 MHz, DMSO-d₆) δ ppm 1.17 (s, 6H) 1.78 (s, 3H) 1.79-1.93 (m, 2H) 2.04 (s, 3H) 2.36 (s, 3H) 2.43 (t, 2H) 6.06 (s, 1H) 6.33-6.55 (m, 4H) 7.03 (dd, 1H) 7.14 (t, 2H) 7.59-7.75 (m, 2H) 10.10 (s, 1H). ¹³C NMR (101 MHz, DMSO-d₆) δ ppm 12.9, 13.7, 13.9, 27.7, 34.2, 35.7, 37.3, 115.5, 115.8, 121.1, 121.1, 123.7, 126.1, 129.2, 130.0, 133.6, 136.3, 138.0, 138.0, 140.8, 148.7, 157.1, 159.5, 160.9, 165.0, 198.2.

### Example 6

### (±) 4-Hydroxy-4-[H]-(4′-fluoro(phenyl)-(E)-retinamide (Compound 17)

Compound 17 was synthesized using General Method B in Example 2. To an ice cold, orange suspension of 4-Oxo-(4′-fluoro(phenyl)-(E)-retinamide (Compound 16) (1 g, 2.45 mmol), methanol (10 mL) and tetrahydrofuran (5 mL) added fresh sodium borohydride (0.123 g, 2.94 mmol) in three portions over 15 minutes. Thereafter, the mixture was stirred for 30 minutes at room temperature. To the resultant yellow solution, acetone (2 mL) was added and stirred for 5 minutes. The reaction mixture was then concentrated under vacuum, treated with ice water, filtered, and dried under vacuum to yield a yellow solid; 0.98 g (97%); mp, 161-163° C.; ¹H NMR (400 MHz, CDCl₃) δ ppm 1.05 (s, 3H) 1.02 (s, 3H) 1.44 (ddd, 1H) 1.54 (d, 1H) 1.62-1.76 (m, 2H) 1.84 (s, 3H) 1.86-1.97 (m, 1H) 2.01 (s, 3H) 2.42 (s, 3H) 4.02 (d, 1H) 5.80 (s, 1H) 6.09-6.19 (m, 2H) 6.19-6.26 (m, 1H) 6.29 (d, 1H) 6.90-7.06 (m, 3H) 7.20 (br 1H) 7.51 (br. s., 2H); ¹H NMR (400 MHz, DMSO-d₆) δ ppm 0.90-1.16 (m, 6H) 1.26-1.39 (m, 1H) 1.50-1.69 (m, 2H) 1.75 (s, 4H) 1.99 (s, 3H) 2.35 (s, 3H) 3.82 (br. s., 1H) 4.67 (br. s., 1H) 6.02 (s, 1H) 6.11-6.27 (m, 2H) 6.31 (d, 1H) 6.39 (d, 1H) 7.01 (dd1H) 7.14 (t, 2H) 7.66 (dd, 2H) 10.07 (s, 1H). ¹³C NMR (101 MHz, DMSO-d₆) δ ppm 13.0, 13.7, 18.9, 28.3, 28.9, 29.2, 34.7, 35.2, 68.7, 115.5, 115.7, 121.1, 121.1, 122.9, 127.8, 130.3, 130.9, 132.3, 136.3, 136.6, 137.9, 138.6, 139.5, 148.9, 157.1, 159.5, 165.0.

### Example 7

### (±) 4-(Imidazolyl-2″3″5″-D₃)-4-(H)-(4′-fluoro(phenyl))-(E)-retinamide (Compound 3)

Compound 3 was synthesized using (±) 4-Hydroxy-4-[H]-(4′-fluoro(phenyl)-(E)-retinamide (Compound 17) (0.73 g, 1.78 mmol) and 1,1′-carbonildiimidazole-d₆ (0.45 g, 2.67 mmol) by following general method C. Yellow solid 0.37 g (44%); mp, sinters at 103° C., melts at 115-117° C.; ¹H NMR (400 MHz, DMSO-d₆) δ ppm 1.11 (s, 3H) 1.08 (s, 3H) 1.42-1.55 (m, 5H) 1.71-1.85 (m, 1H) 2.01 (s, 3H) 2.02-2.12 (m, 1H) 2.29-2.40 (m, 3H) 4.74 (t, 1H) 6.03 (s, 1H) 6.24-6.32 (m, 2H) 6.32-6.45 (m, 2H) 7.01 (dd, 1H) 7.08-7.19 (m, 2H) 7.60-7.71 (m, 2H) 10.10 (s, 1H); ²H NMR (146 MHz,0.5% DMSO-d₆ in DMSO) S ppm 6.97 (br s, 1D) 7.13 (br s, 1D) 7.71 (br s, 1D); ¹³C NMR (101 MHz, DMSO-d₆) δ ppm 13.0, 13.7, 18.8, 28.0, 28.2, 29.1, 34.7, 35.0, 39.3, 39.5, 39.7, 39.9, 40.1, 40.4, 40.6, 57.6, 115.5, 115.7, 121.0, 121.1, 123.2, 125.6, 126.8, 130.2, 131.6, 136.3, 136.3, 137.0, 138.2, 139.1, 144.1, 148.8, 157.1, 159.5, 165.0; HRMS calcd 463.2946 (C₂₉H₃₁D₃FN₃O H+) found 463.2949.

### Example 8

### (±) 4-(Imidazolyl)-4-(D)-(4′-fluoro(phenyl))-(E)-retinamide (Compound 4)

Compound 4 was synthesized using (±) 4-Hydroxy-4-[D]-(4′-fluoro(phenyl)-(E)-retinamide (Compound 18) (0.5 g, 1.29 mmol) and 1,1′-carbonildiimidazole (0.31 g, 1.93 mmol) by following general method C. Yellow solid 0.27 g (47%); mp, 165-167° C.; ¹H NMR (400 MHz, DMSO-d₆) δ ppm 1.09 (s, 3H) 1.12 (s, 3H) 1.51 (s, 4H) 1.73-1.82 (m, 1H) 2.02 (s, 3H) 2.03-2.09 (m, 1H) 2.36 (s, 3H) 6.03 (s, 1H) 6.25-6.45 (m, 4H) 6.93 (s, 1H) 7.02 (dd, 1H) 7.08-7.18 (m, 3H) 7.63 (s, 1H) 7.67 (dd, 2H) 10.08 (s, 1H); ²H NMR (146 MHz,0.5% DMSO-d₆ in DMSO) δ ppm 4.69 (br s, 1D); ¹³C NMR (101 MHz, DMSO-d₆) δ ppm 12.4, 13.1, 18.2, 27.4, 27.7, 28.6, 34.1, 34.5, 115.0, 115.2, 118.2, 120.5, 120.5, 122.6, 125.2, 126.3, 128.2, 129.6, 131.0, 135.7, 135.8, 136.4, 136.7, 137.7, 138.5, 143.4, 148.3, 156.5, 158.9, 164.4; HRMS calcd 461.2821 (C₂₉H₃₃DFN₃O H+) found 461.2820.

### Example 9

### (±) 4-(Imidazolyl-2″3″5″-D₃)-4-(D)-(4′-fluoro(phenyl))-(E)-retinamide (Compound 5)

Compound 5 was synthesized using (±) 4-Hydroxy-4-[D]-(4′-fluoro(phenyl)-(E)-retinamide (Compound 18) (0.5 g, 1.22 mmol) and 1,1′-carbonildiimidazole-d₆ (0.31 g, 1.82 mmol) by following general method C. Yellow solid 0.27 g (47%); mp, sinters at 125° C., melts at 131° C.; ¹H NMR (400 MHz, DMSO-d₆) δ ppm 1.09 (s, 3H) 1.13 (s, 3H) 1.45-1.51 (m, 2H) 1.53 (s, 3H) 1.80-1.87 (m, 1H) 2.02 (s, 3H) 2.36 (s, 3H) 6.09 (s, 1H) 6.19-6.32 (m, 2H) 6.32-6.46 (m, 2H) 7.01 (dd, 1H) 7.07-7.26 (m, 2H) 7.56-7.82 (m, 2H) 10.24 (s, 1H); ²H NMR (146 MHz,0.5% DMSO-d₆ in DMSO) δ ppm 4.90 (br s, 1D) 6.95-7.55 (br s, 2D) 7.61 (br s, 1D). ¹³C NMR (101 MHz, DMSO-d₆) δ ppm 13.0, 13.7, 18.7, 27.3, 28.1, 29.0, 34.5, 34.8, 115.5, 115.7, 121.0, 121.1, 123.4, 123.8, 126.5, 130.1, 131.8, 136.4, 137.2, 138.1, 139.3, 145.8, 148.7, 157.1, 159.4, 165.0; HRMS calcd 464.3009 (C₂₉H₃₀D₄FN₃O H+) found 434.3016.

### Example 10

### (±) 4-Imidazolyl-4(H)-(4′-fluoro(phenyl-2′3′5′6′-D₄))-(E)-retinamide (Compound 6)

Compound 6 was synthesized using (±) 4-Hydroxy-4-[H]-(4′-fluoro(phenyl-2′3′5′6′-D₄)-(E)-retinamide (Compound 20) (0.5 g, 1.21 mmol) and 1,1′-carbonildiimidazole (0.29 g, 1.81 mmol) by following general method C to yield a yellow solid 0.29 g (51%); mp, sinters at 105° C., melts at 113-115° C.; ¹H NMR (400 MHz, DMSO-d₆) δ ppm 1.08 (s, 3H) 1.11 (s, 3H) 1.45-1.51 (m, 5H) 1.74-1.82 (m, 1H) 2.01 (s, 3H) 2.05 (d, 1H) 2.35 (s, 3H) 4.74 (br. s., 1H) 6.02 (s, 1H) 6.28 (m, 2H) 6.33-6.43 (m, 2H) 6.97 (s, 1H) 6.99-7.06 (m, 1H) 7.14 (s, 1H) 7.72 (br. s., 1H) 10.08 (s, 1H); ²H NMR (146 MHz,0.5% DMSO-d₆ in DMSO) δ ppm 7.15 (br s, 2D) 7.67 (br s, 2D); ¹³C NMR (101 MHz, DMSO-d₆) δ ppm 13.0, 13.7, 18.8, 28.0, 28.2, 29.1, 34.7, 35.0, 57.6, 118.9, 123.2, 125.6, 126.8, 128.2, 130.2, 131.6, 136.2, 137.0, 137.2, 138.2, 139.1, 144.1, 148.8, 157.0, 159.4, 165.0; 464.3009 (C₂₉H₃₀D₄FN₃O H+) found 464.3010.

### Example 11

### (±) 4-(Imidazolyl-2″3″5″-D₃)-4-(H)-(4′-fluoro(phenyl-2′3′5′6′-D₄))-(E)-retinamide (Compound 7)

Compound 7 was synthesized using (+) 4-Hydroxy-4-[H]-(4′-fluoro(phenyl-2′3′5′6′-D₄)-(E)-retinamide (Compound 20) (0.5 g, 1.21 mmol) and 1,1′-carbonildiimidazole-d₆ (0.31 g, 1.81 mmol) by following general method C to yield a yellow solid 0.15 g (27%); mp, sinters at 105° C., melts at 113-115° C.; ¹H NMR (400 MHz, DMSO-d₆) δ ppm 1.08 (s, 3H) 1.11 (s, 3H) 1.47 (d, 2H) 1.48-1.51 (m, 3H) 1.77 (dd, 1H) 2.01 (s, 3H) 2.06 (d, 1H) 2.35 (s, 3H) 4.73 (t, 1H) 6.03 (s, 1H) 6.27-6.45 (m, 4H) 7.01 (dd, 1H) 10.10 (s, 1H); ²H NMR (146 MHz,1% DMSO-d₆ in DMSO) δ ppm 6.91 (br s, 1D) 6.99-7.31 (br s, 3D) 7.5-7.89 (br s, 3D). ¹³C NMR (101 MHz, DMSO-d₆) δ ppm 13.0, 13.7, 18.4, 18.7, 18.8, 27.9, 28.1, 28.3, 29.1, 34.6, 34.7, 35.1, 57.4, 123.2, 125.8, 126.9, 130.2, 131.6, 136.1, 137.0, 138.2, 139.0, 143.9, 148.8, 157.0, 159.4, 165.0; HRMS calcd 467.3198 (C₂₉H₂₇D₇FN₃O H+) found 467.30198.

### Example 12

### (±) 4-(Imidazolyl)-4-(D)-(4′-fluoro(phenyl-2′3′5′6′-D₄))-(E)-retinamide (Compound 8)

Compound 8 was synthesized using (+) 4-hydroxy-4-[D]-(4′-fluoro(phenyl-2′3′5′6′-D₄)-(E)-retinamide (Compound 21) (0.5 g, 1.21 mmol) and 1,1′-carbonildiimidazole (0.29 g, 1.81 mmol) by following general method C. to give a yellow solid 0.24 g (42%); mp, sinters at 95° C., melts at 112° C.; ¹H NMR (400 MHz, DMSO-d₆) δ ppm 1.08 (s, 3H) 1.11 (s, 3H) 1.45-1.52 (m, 5H) 1.74-1.80 (m, 1H) 2.01 (s, 3H) 2.03-2.08 (m, 1H) 2.35 (s, 3H) 6.03 (s, 1H) 6.20-6.47 (m, 4H) 6.90-6.95 (m, 1H) 7.01 (dd, 1H) 7.10 (m, 1H) 7.62 (s, 1H) 10.10 (s, 1H); ²H NMR (146 MHz,0.5% DMSO-d₆ in DMSO) δ ppm 4.69 (br s, 1D) 7.15 (br s, 2D) 7.67 (br s, 2D). ¹³C NMR (101 MHz, DMSO-d₆) δ ppm 13.0, 13.7, 18.8, 27.9, 28.1, 28.2, 29.1, 34.7, 35.0, 118.7, 123.2, 125.7, 125.8, 126.9, 128.7, 130.2, 131.6, 136.2, 137.0, 137.3, 138.2, 139.0, 144.0, 144.0, 148.8, 157.0, 159.4, 165.0; HRMS calcd 465.3072 (C₂₉H₂₉D₅FN₃O H+) found 465.3075.

### Example 13

### (±) 4-(Imidazolyl-2″3″5″-D₃)-4-(D)-(4′-fluoro(phenyl-2′3′5′6′-D₄))-(E)-retinamide (Compound 9)

Compound 9 was synthesized using (+) 4-hydroxy-4-[D]-(4′-fluoro(phenyl-2′3′5′6′-D₄)-(E)-retinamide (Compound 21) (0.5 g121 mmol) and 1,1′-carbonildiimidazole-d₆ (0.3 g, 1.81 mmol) by following general method C to give a yellow solid 0.135 g (24%); mp, sinters at 95° C., melts at 108° C.; ¹H NMR (400 MHz, DMSO-d₆) δ ppm 1.08 (s, 3H) 1.11 (s, 3H) 1.46-1.49 (m, 2H) 1.50 (s, 3H) 1.74-1.80 (m, 1H) 2.01 (s, 3H) 2.03-2.08 (m, 1H) 2.32-2.37 (s, 3H) 6.03 (s, 1H) 6.28 (s, 2H) 6.33-6.44 (m, 2H) 7.01 (dd, 1H) 10.05-10.12 (m, 1H); ²H NMR (146 MHz,0.5% DMSO-d₆ in DMSO) δ ppm 4.70 (br s, 1D) 6.94 (br s, 1D) 7.10-7.32 (br s, 3D) 7.40-7.92 (br s, 3D). ¹³C NMR (101 MHz, DMSO-d₆) δ ppm 13.0, 13.7, 18.8 27.98, 28.1, 28.2, 29.1, 34.7, 35.1, 39.3, 39.5, 39.7, 39.9, 40.1, 40.3, 40.6, 123.2, 125.7, 125.8, 126.2, 130.2, 131.6, 136.2, 137.0, 138.3, 139.0, 144.0, 148.8, 157.0, 159.4, 165.0; HRMS calcd 468.3260 (C₂₉H₂₆D₈FN₃O H+) found 468.3264.

### Example 14

### (±) 4-Hydroxy-4-[D]-(4′-fluoro(phenyl)-(E)-retinamide (Compound 18)

Compound 18 was synthesized using 4-Oxo-(4′-fluoro(phenyl)-(E)-retinamide (Compound 16) (2 g, 4.91 mmol) and sodium borodueteride (0.28 g, 5.89 mmol) by following general method B. Yellow solid 1.90 g (94%); mp, 173-174° C.; ¹H NMR (400 MHz, DMSO-d₆) δ ppm 0.99 (s, 3H) 1.02 (s, 3H) 1.28-1.38 (m, 1H) 1.49-1.68 (m, 2H) 1.68-1.81 (m, 4H) 1.99 (s, 3H) 2.35 (s, 3H) 4.64 (s, 1H) 6.01 (s, 1H) 6.14-6.28 (m, 2H) 6.31 (d, 1H) 6.39 (d, 1H) 7.01 (dd, 1H) 7.09-7.19 (m, 2H) 7.61-7.71 (m, 2H) 10.07 (s, 1H); ²H NMR (146 MHz,0.5% DMSO-d₆ in DMSO) δ ppm 3.78 (br s, 1 D). ¹³C NMR (101 MHz, DMSO-d₆) δ ppm 13.0, 13.7, 18.9, 28.2, 28.7, 29.2, 34.7, 35.2, 39.3, 39.5, 39.7, 39.9, 40.1, 40.4, 40.6, 115.5, 115.8, 121.1, 121.1, 122.9, 127.8, 130.3, 130.9, 132.3, 136.3, 136.6, 137.9, 138.6, 139.5, 148.9, 157.1, 159.5, 165.1.

### Example 15

### 4-Oxo-(4′-fluoro(phenyl-2′3′5′6′-D₄)-(E)-retinamide (Compound 19)

Compound 19 was synthesized using 4-Oxo-ATRA (Compound 15) (6 g, 19.1 mmol) and 4-fluoroaniline-2,3,5,6-d₄ (2.42 g, 21.0 mmol) by following general method A to give an orange solid 6.60 g (84%); mp 156-159° C.; ¹H NMR (400 MHz, DMSO-d₆) δ ppm 1.17 (s, 6H) 1.77 (s, 3H) 1.78-1.84 (m, 2H) 2.04 (s, 3H) 2.36 (s, 3H) 2.43 (t, 2H) 6.05 (s, 1H) 6.29-6.58 (m, 4H) 7.03 (dd, 1H) 10.11 (s, 1H); ²H NMR (146 MHz,0.5% DMSO-d₆ in DMSO) δ ppm 7.14 (br s, 2 D), 7.66 (br s, 2 D). ³C NMR (101 MHz, DMSO-d₆) δ ppm 12.9, 13.7, 13.9, 27.7, 34.2, 35.7, 37.3, 123.7, 126.1, 129.2, 130.0, 133.6, 136.1, 137.9, 138.0, 140.7, 148.6, 157.0, 159.4, 160.92, 164.9, 198.2.

### Example 16

### (±) 4-Hydroxy-4-[H]-(4′-fluoro(phenyl-2′3′5′6′-D₄)-(E)-retinamide (Compound 20)

Compound 20 was synthesized using 4-Oxo-(4′-fluoro(phenyl-2′3′5′6′-D₄)-(E)-retinamide (Compound 19) (2.5 g, 6.07 mmol) and sodium borohydriride (0.28 g, 7.29 mmol) by following general method B to give a yellow solid 2.35 g (94%); mp, sinters at 120° C., melts at 153-156° C.; ¹H NMR (400 MHz, DMSO-d₆) δ ppm 0.99 (s, 3H) 1.02 (s, 3H) 1.30-1.36 (m, 1H) 1.52-1.62 (m, 2H) 1.75 (s, 4H) 1.99 (s, 3H) 2.35 (s, 3H) 3.81 (d, 1H) 4.66 (d, 1H) 6.02 (s, 1H) 6.14-6.27 (m, 2H) 6.31 (d, 1H) 6.39 (d, 1H) 7.01 (dd, 1H) 10.07 (s, 1H); ²H NMR (146 MHz,0.5% DMSO-d₆ in DMSO) δ ppm 7.12 (br s, 2 D), 7.65 (br s, 2 D); ¹³C NMR (101 MHz, DMSO-d₆) δ ppm 13.0, 13.7, 18.9, 28.3, 28.9, 29.2, 34.7, 35.2, 39.3, 39.5, 39.7, 39.98, 40.1, 40.4, 40.60, 68.7, 123.0, 127.8, 130.3, 130.9, 132.3, 136.2, 136.6, 137.9, 138.6, 139.5, 148.9, 157.0, 159.4, 165.0.

### Example 17

### (±) 4-Hydroxy-4-[D]-(4′-fluoro(phenyl-2′3′5′6′-D₄)-(E)-retinamide (Compound 21)

Compound 21 was synthesized using 4-Oxo-(4′-fluoro(phenyl-2′3′5′6′-D₄)-(E)-retinamide (Compound 19) (2.5 g, 6.07 mmol) and sodium borodueteride (0.3 g, 7.9 mmol) by following general method B to give a yellow solid 2.23 g (87%); mp, sinters at 95° C., melts at 101-103° C.; ¹H NMR (400 MHz, DMSO-d₆) δ ppm 0.99 (s, 3H) 1.02 (s, 3H) 1.30-1.36 (m, 1H) 1.50-1.62 (m, 2H) 1.70-1.77 (m, 4H) 1.99 (s, 3H) 2.35 (s, 3H) 4.64 (br. s., 1H) 6.02 (s, 1H) 6.10-6.24 (m, 2H) 6.25-6.34 (m, 1H) 6.39 (d, 1H) 7.00 (dd, 1H) 10.08 (s, 1H); ²H NMR (146 MHz,3% DMSO-d₆ in DMSO) δ ppm 3.73 (br s, 1D) 7.16 (br s, 2 D) 7.68 (br s, 2 D); ¹³C NMR (101 MHz, DMSO-d₆) δ ppm 13.0, 13.7, 18.9, 28.2, 28.7, 28.9, 29.2, 34.7, 35.2, 123.0, 127.8, 130.3, 130.9, 132.3, 132.3, 136.2, 136.6, 137.9, 138.6, 139.5, 148.9, 157.0, 159.4, 165.0

### Example 18: Cellular Antiproliferative Activities Against Two TNBC Cell Lines

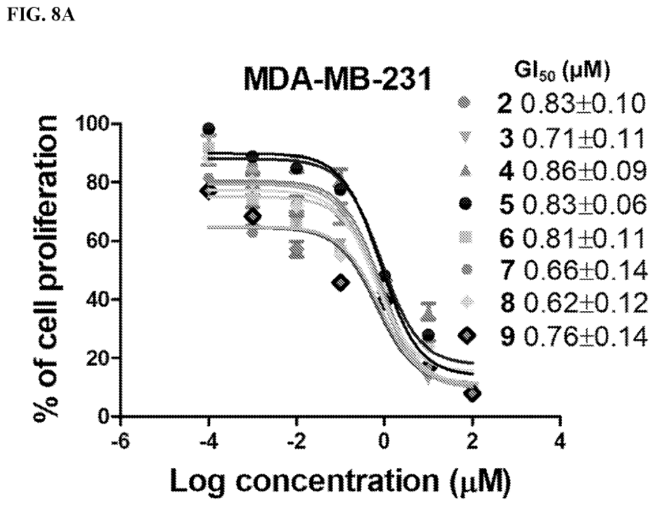

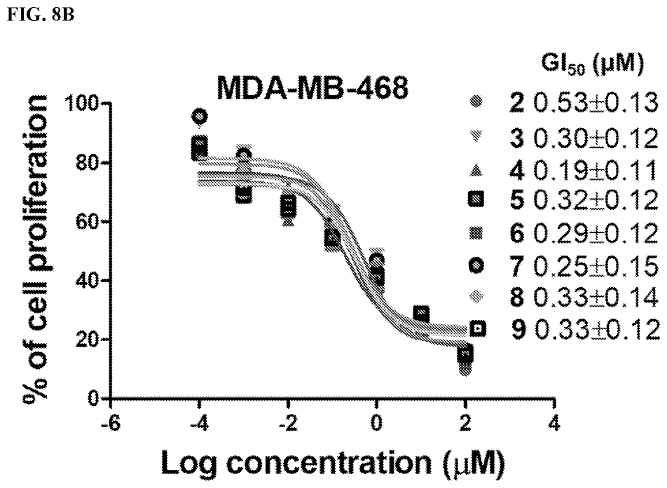

Compounds 3 to 9 were evaluated over a range of concentrations to examine their antiproliferative activities against two TNBC cell lines, MDA-MB-231 and MDA-MB-468 using the well-established 3-(4,5-dimethylthiazolyl-2)-2,5-diphenyl tetrazolium bromide (MTT) assay.^{48,55,56} The parent non-deuterated VNLG-152R (Compound 2) was used as the positive control. As presented in FIG. 8A, the GI₅₀ values (620-830 nM range) of the deuterated Compounds 3 to 9 against the MDA-MB-231 cell are identical to the GI₅₀ value of the parent protio VNLG-152R (Compound 2). In contrast, as shown in FIG. 8B, the MDA-MB-468 cell appear to be more sensitive (GI₅₀ values: 190-530 nM range) to the deuterated compounds (1.61-2.79-folds) compared to the parent non-deuterated VNLG-152R (Compound 2). The reason for this difference is not understood at this time. The applicant hypothesizes that the enhanced diffusion of these deuterated molecules across the cell membrane of this cell line may result in enhanced intracellular concentrations and inhibition of its proliferation. In FIGS. 8A and 8B, the dose-response curves were generated from MTT assays after 6-day exposure of different concentrations of the compounds. Each point is a mean of replicates from three independent experiments. The GI₅₀ values were determined from the dose-response curves by nonlinear regression analysis using GraphPad Prism.

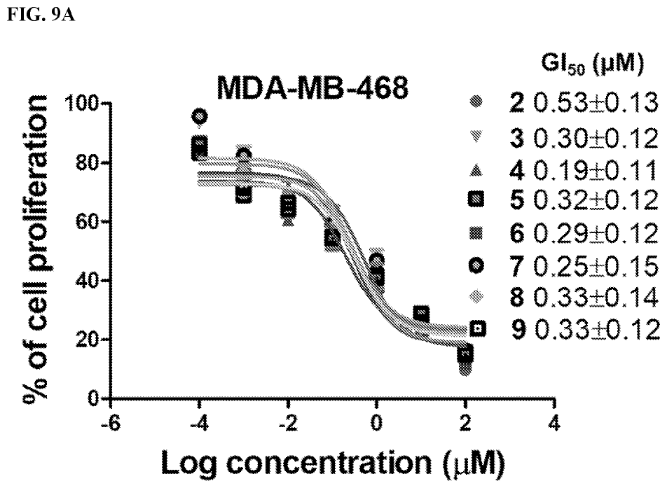

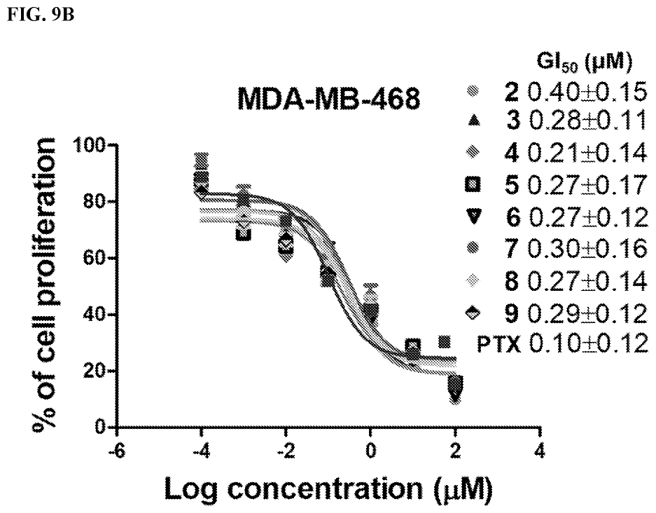

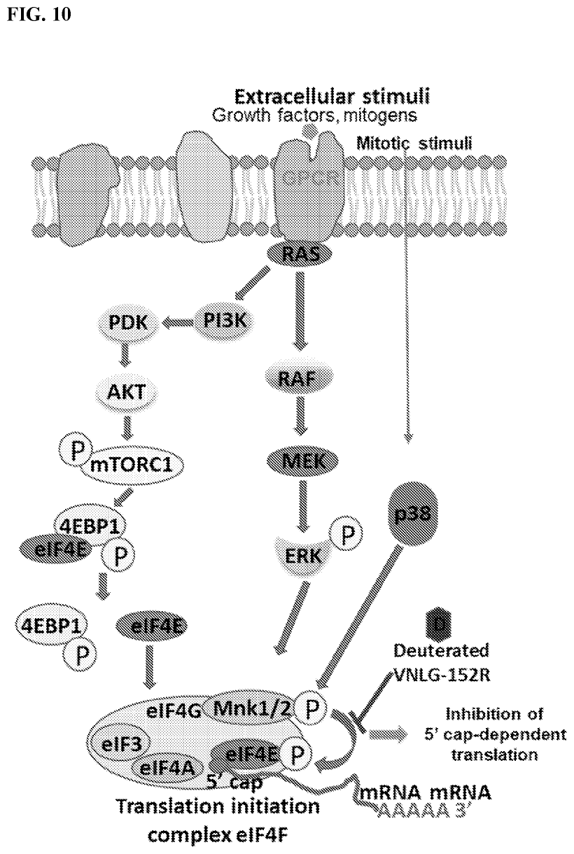

Additionally, a similar evaluation was performed in which Compounds 3-9 were evaluated in the same manner as discussed above with respect to FIGS. 8A and 8B. However, the Compounds 3-9 were additionally compared to paclitaxel (PTX). As illustrated in FIGS. 9A and 9B, the deuterated analogs (Compounds 3-9) were either better or equipotent to the non-deuterated VNLG-152R (Compound 2) in in vitro antiproliferative activities against MDA-MB-231 and MDA-MB-468 TNBC cell lines.

### Example 19: Cellular Antiproliferative Activities Against Two Non-TNBC Cell Lines

Compounds 2, 4, 8 and 9, as well as paclitaxel (PTX) were evaluated over a range of concentrations to examine their antiproliferative activities against two non-TNBC cell lines, MCF7 and SKBR3using the well-established 3-(4,5-dimethylthiazolyl-2)-2,5-diphenyl tetrazolium bromide (MTT) assay.^{48,55,56} The parent non-deuterated VNLG-152R (Compound 2) was used as the positive control.

As presented in Table 1 below, the GI₅₀ values of the deuterated Compounds 4, 8 and 9 against the MCF7 cell are identical to the GI₅₀ value of the parent protio VNLG-152R (Compound 2). In contrast, the SKBR3 cell appears to be more sensitive to the deuterated compounds compared to the parent non-deuterated VNLG-152R (Compound 2). The reason for this difference is not understood at this time. The applicant hypothesizes that the enhanced diffusion of these deuterated molecules across the cell membrane of this cell line may result in enhanced intracellular concentrations and inhibition of its proliferation. Each data point is a mean of replicates from three independent experiments.

TABLE 1

| Cell lines↓ | Compound 2 | Compound 4 | Compound 8 | Compound 9 | Paclitaxel |
|---|---|---|---|---|---|
| MCF7 | 0.35 ± 0.11 | 0.23 ± 0.25 | 0.33 ± 0.14 | 0.22 ± 0.13 | 0.06 ± 0.12 |
| SKBR3 | 0.26 ± 0.12 | 0.14 ± 0.23 | 0.11 ± 0.20 | 0.10 ± 0.14 | 0.06 ± 0.12 |

### Example 20: Effects of VNLG-152R (Compound 2) and its Deuterated Analogs on Mnk1, eIF4E, peIF4E, Cyclin D1, Bcl2 and Bax

The applicant had previously reported that the antiproliferative effects of Compound 1 and our other Mnk1/2 degraders in breast cancer cells was due to degradation of Mnk1/2 with consequent depletion of peIF4E and modulation of the downstream molecular targets.^{48,55,56} Thus, the deuterated analogs were further examined for their effects on the degradation of Mnk1 and the other related downstream molecular targets in both MDA-MB-231 and MDA-MB-468 TNBC cells by Western blotting analysis.

### Cell Culture and Western Blotting

The human breast cancer cell lines, MDA-MB-231, MDA-MB-468 were procured from ATCC (Manassas, VA) and cultured in the recommended media supplemented with 10% heat-inactivated standard fetal bovine serum (FBS, GIBCO) and 1% penicillin-streptomycin (10,000 U/ml, Life Technologies) at 37° C. and 5% CO₂. Primary antibodies against Mnk1, eIF4E, p-eIF4E, Cyclin D₁, BCL2, BAX, β-actin, and secondary RP-conjugated anti-rabbit used in the study were procured from Cell Signaling Technology, USA. Cells were lysed with radioimmunoprecipitation assay (RIPA) buffer supplemented with 1× protease inhibitors (Roche, Indianapolis, IN, USA), phosphatase inhibitors (Thermo Scientific, Waltham, MA, USA), 1 mmol/L EDTA and 1 mmol/L PMSF (Sigma) and immunoblotting analyses were performed as described previously.^{48,64}

### Cell Proliferation Analysis

Cell proliferation assay was performed using MTT (3-(4,5-dimethylthiazol-2-yl)-2,5-diphenyltetrazolium bromide) compounds as described previously.^{51,56} Briefly, 2500 cells/well (MDA-MB-231 and MDA-MB-468) were seeded in 96 well plate for 24 h and treated with the indicated concentrations of VNLG for 2nd and 5th day in a span of eight days. Growth inhibitory concentration (GI₅₀) was calculated based on a non-linear regression curve fit using GraphPad prism 5.0 software (La Jolla, CA).

### Results

The non-deuterated VNLG-152R (Compound 2) and the vehicle, DMSO were used as positive and negative controls, respectively. Equal protein concentrations from breast cancer cells treated with the compounds at different concentration (5, 10 and 20 M) for 24 h were separated by SDS-PAGE and Western blots probed with the respective protein antibodies. Vehicle treated cells were included as a control, and all blots were reprobed for R-actin for loading control.

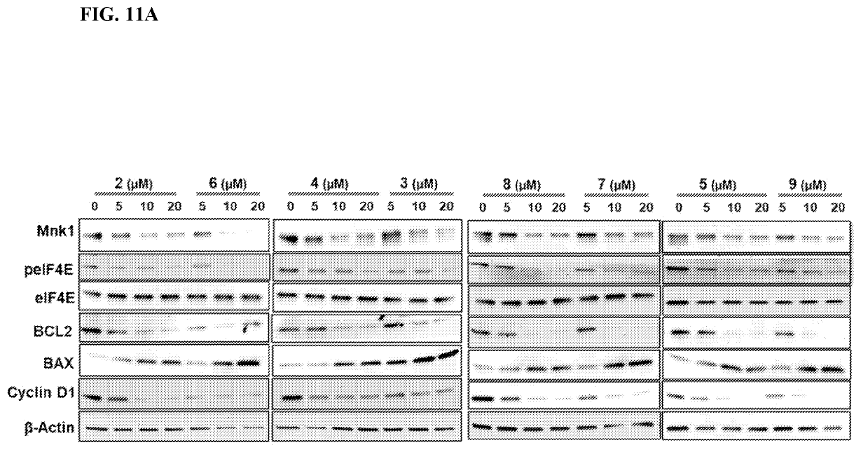

As shown in FIG. 11A, in the MDA-MB-231 cells, VNLG-152R (Compound 2) and the deuterated analogs significantly and dose-dependently reduced the expressions of Mnk1, and peIF4E, without noticeable effects on the expression of total eIF4E and the house keeping protein, β-actin. FIG. 11A also shows that the compounds also cause significant depletion of the downstream target, cyclin D1 and induction of apoptosis via significant downregulation of antiapoptotic Bcl-2 and upregulation of proapoptotic Bax. A similar phenomenon was also observed in MDA-MB-468 cells (FIG. 11B). As expected, the potency of the non-deuterated VNLG-152R (Compound 2) was equipotent with the deuterated Compounds 3 to 9.

### Example 21: Pharmacokinetics Parameters of Deuterated Compounds 4, 8, and 9 in Mouse are Superior to Those of Non-Deuterated VNLG-152R (Compound 2)

As described above, the antiproliferative effects and their mechanisms of action of the deuterated Compounds 3-9 were for the most part at par with the biological activities of the parent non-deuterated VNLG-152R (Compound 2). Thus, the deuterated compounds met all criteria of our objective to develop new potent Mnk1/2 degraders with improved pharmacokinetics. Hence, although the pharmacokinetics of VNLG-152R (Compound 2) has previously been reported,⁶⁵ the plasma pharmacokinetics (PK) of Compounds 3-9 were compared head-to-head, that is, evaluated under the same study conditions, with VNLG-152R (Compound 2) after oral administration in female CD-1 mice.

### In Vivo Pharmacokinetics (PK) Studies

The PK study was performed by the Certified Research Organization (CRO), Aragen Life Sciences Private Limited (Aragen), Hyderabad, India. All procedures of the present study were in accordance with the guidelines provided by the Committee for the Purpose of Control and Supervision of Experiments on Animals (CPCSEA) as published in The Gazette of India, Jan. 7, 2010. CPCSEA renewal number for institute is 1125/PO/Rc/S/CPCSEA on 13Feb. 2014. Institutional Animal Ethics Committee (IAEC) approval has been taken to initiate the study (IAEC Approval Number: B-011A). The details are presented in the Supplementary Materials.

To minimize the number of mice used in the PK study using a validated LC/MS/MS analysis, both individual (discrete) (set #1: Compound 5, EM=463.2937 and set #2: Compound 7, EM=463.2937) and cassette (set #3: Compounds 2, 3, and 7; EMs=459.2686, 462.2874 and 466.3125, respectively, and set #4: Compounds 4, 8, and 9; EMs=460.2749, 464.2999 and 467.3188, respectively) dosing were used.^{59,66} The four sets studied were dictated by the exact molecular masses of the compounds as indicated above and in FIG. 3.

All experiments were carried out in at least triplicates and are expressed as mean±S.E. where applicable. Treatments were compared to controls using the student's t-test with either GraphPad Prism or Sigma Plot. Differences between groups were considered statistically significant at P<0.05.

### Results

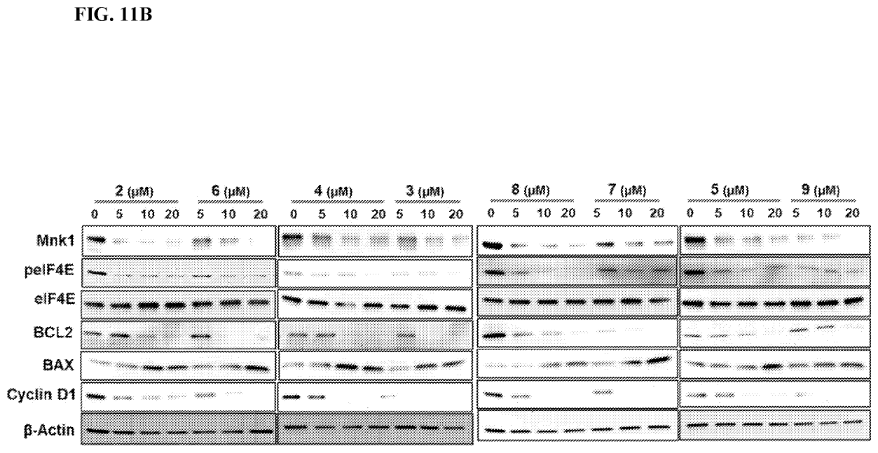

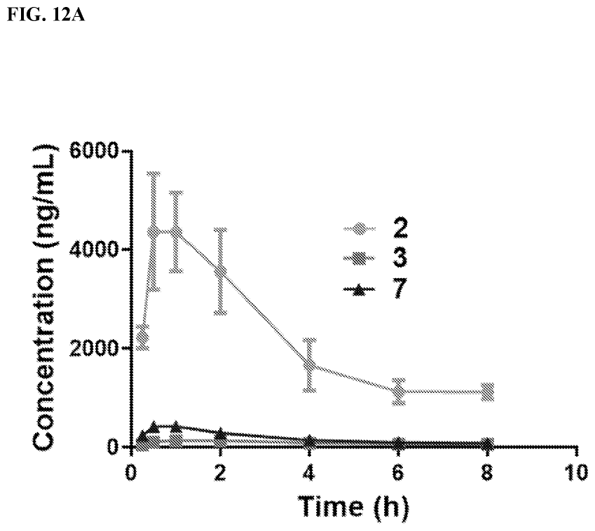

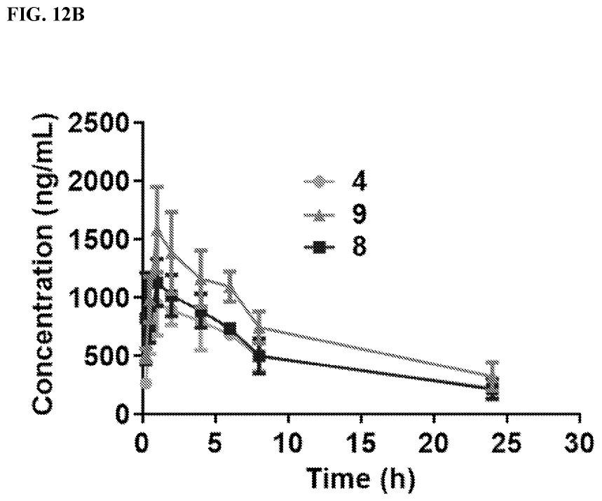

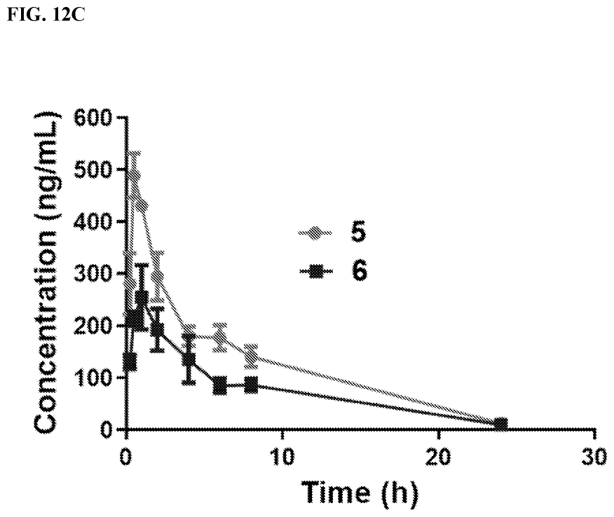

The plasma PK profiles were obtained after single oral (PO) dosing of the compounds in mice. As shown in FIGS. 12A-12C, the plasma concentration versus time profiles shows that the non-deuterated VNLG-152R (Compound 2) and two deuterated Compounds 4 and 8 are rapidly cleared from systemic circulation as they could not be detected after 8 hours following their administrations. It is also notable that whereas VNLG-152R (Compound 2) exhibited the best Cₘₐₓ and AUC, Compounds 4 and 8 exhibited very low systemic exposures.

The significant PK parameters are presented in Table 2. The total scores (8 to 26 and overall ranking) in Table 2 are the sum of the rankings of the four PK parameters, including Cₘₐₓ, T_{1/2}, AUC and MRT. In Table 2, data are presented as the mean of values obtained from three female CD-1 mice. Cₘₐₓ is the maximum observed plasma concentration. Tₘₐₓ is the time to maximum concentration. T_{1/2} is the elimination half-life. AUC represents AUC (0-∞), which is the area under the concentration-time curve from time of dosing extrapolated to infinity. MRT refers to mean residence time. The number in parentheses indicate the rank order from best (1) to worst (8). The total scores (8 to 26 and overall ranking) are the sum of the rankings of the four PK parameters, including Cₘₐₓ, T_{1/2}, AUC and MRT

TABLE 2
Pharmacokinetic parameters of compound 1 and its deuterated analogs
(Compounds 3-9) in female CD-1 mice after a single p.o. dose 10 mg/kg

| | Cₘₐₓ | | T_{1/2} | AUC | MRT | Total |
|---|---|---|---|---|---|---|
| Compound | (ng/mL) | Tₘₐₓ (h) | (h) | (ng · h/mL) | (h) | Score [Rank] |
| 2 | 4651.88 (1) | 0.83 | 3.61 (8) | 22764.75 (1) | 3.02 (7) | 17 [4^{th}] |
| 3 | 151.17 (7) | 1.33 | 8.39 (4) | 1737.92 (7) | 3.57 (6) | 24 [6^{th}] |
| 4 | 1031.65 (4) | 2.50 | 12.05 (1) | 17568.45 (3) | 9.06 (1) | 9 [2^{nd}] |
| 5 | 500.72 (5) | 0.67 | 4.27 (6) | 2703.99 (5) | 5.49 (4) | 20 [5^{th}] |
| 6 | 272.72 (8) | 0.83 | 4.90 (5) | 1727.34 (7) | 4.63 (5) | 25 [7^{th}] |
| 7 | 454.97 (6) | 0.83 | 4.34 (6) | 2061.99 (6) | 2.89 (8) | 26 [8^{th}] |
| 8 | 1361.22 (3) | 0.75 | 10.20 (2) | 15536.70 (4) | 8.48 (3) | 12 [3^{rd}] |
| 9 | 1615.81 (2) | 2.67 | 10.28 (2) | 22571.5 (2) | 8.92 (2) | 8 [1^{st}] |

Three deuterated Compounds 4, 8, and 9 with total scores of 9 (2^{nd}), 12 (3^{rd}) and 8 (1^{st}), respectively, are superior to the non-deuterated VNLG-152R (Compound 2) with a total score of 17 (4^{th}). Notably, compared to the non-deuterated VNLG-152R (Compound 2), Compound 16 with only a C4-deuterium exhibited the longest half-life (T_{1/2}) which was extended by 8.44 hours (3.61 hours versus 12.05 hours), and the mean residence time (MRT) was extended by 6.04 hours (3.02 hours versus 9.06 hours). It is unclear why the AUC of all the deuterated compounds were lower than the AUC of non-deuterated VNLG-152R (Compound 2).

It should be noted that deuterium substitution does not always result in slowed metabolism.⁶⁷ For example, a deuterated form of paroxetine (CTP-347) has demonstrated increased metabolism when compared with the non-deuterated form.⁶⁶ It is also important to state here that deuteration as a strategy to alter pharmacokinetics requires the importance of understanding the systemic clearance mechanism and knowing the identity of the metabolic enzymes involved, the extent to which they contribute to metabolic clearance, and the extent to which metabolism contributes to the systemic clearance.^{66,68}

### Example 22: Anti-Tumor Activity of Deuterated Compounds 4, 8, and 9 in Mouse are Superior to Those of Non-Deuterated VNLG-152R (Compound 2)

After confirming the improved pharmacokinetic parameters of deuterated compounds 4, 8, and 9, the anti-tumor activity of these compounds was tested in xenograft models of MDA-MB-231 and MDA-MB-468. In both models, the mice for each group were treated with either vehicle (control) or the compounds, each at 20 mg/kg, PO, 5 days/week.

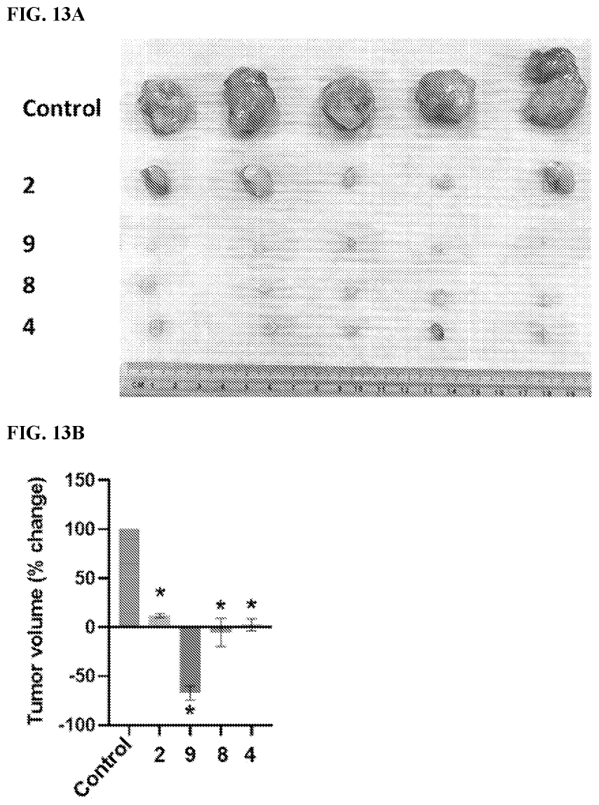

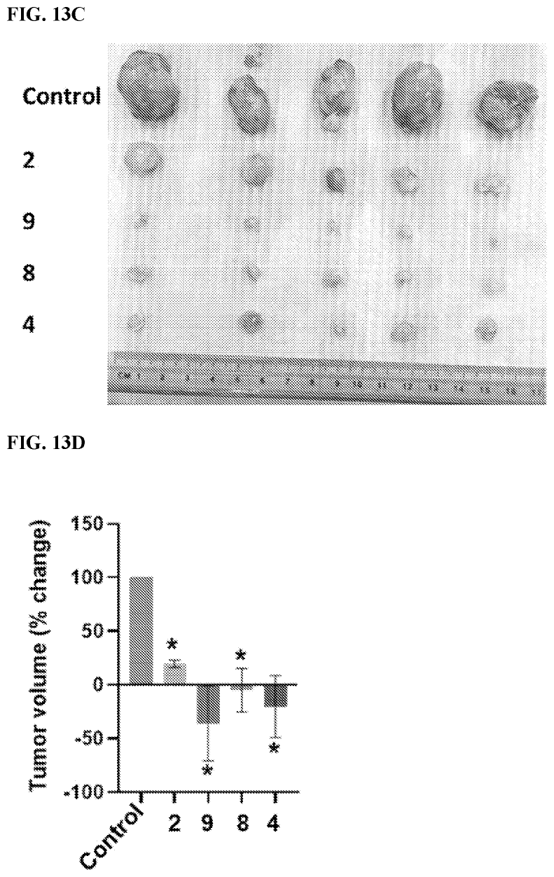

As shown in FIGS. 13A and 13B, in the MDA-MB-213 model, whereas the non-deuterated compound VNLG-152R reduced tumor growth by 87%, the deuterated Compounds 4 and 8 reduced tumor growth by 95%, while the deuterated Compound 9 caused tumor regression by 67%. *=p<0.0001.

Similarly, as shown in FIGS. 13C and 13D, in the MDA-MB-468 model, whereas the non-deuterated compound VNLG-152R reduced tumor growth by 81%, the deuterated Compound 8 reduced tumor growth by 93%, while the deuterated Compounds 4 and 9 caused tumor regressions by 6.6% and 37%, respectively. *=p<0.0001.

In both the MDA-MB-231 and MDA-MB-468 models, body weights of the mice in each group increased from ˜19 g to 21.5 g, indicating that the compounds were safe (well-tolerated) at the effective doses, i.e., no apparent host toxicity.

### Example 23: Molecular Mechanism Studies of VNLG-152R

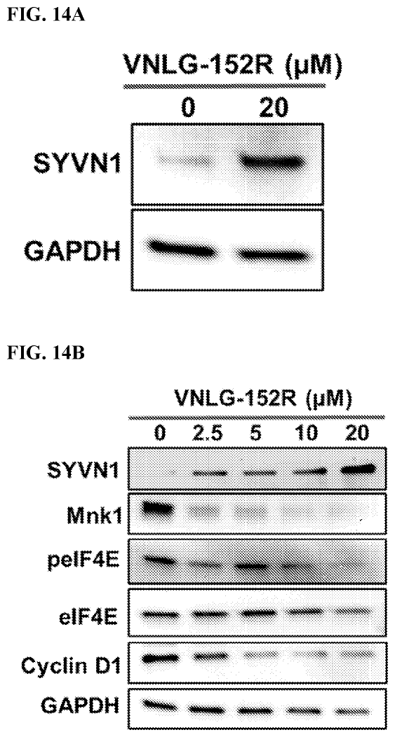

After confirming the anti-tumor effect of VNLG-152R, the molecular mechanism of VNLG-152R was studied, in order to help identify potential new molecular targets for treatment. Using an established procedure^{70,71}, a label-free proteomics analysis was conducted. MDA-MB-231 cells were treated for 24 hours with VNLG-152R in the concentrations indicated in FIGS. 14A and 14B, followed by Western blotting of whole cell lysates.

These studied revealed that VNLG-152R significantly upregulated (FC>3, FDR adjusted p<0.0005) E3 ubiquitin-protein ligase, synoviolin 1 (SYVN1, a.k.a. DER3, HRD1). See FIGS. 14A and 14B. Additionally, as shown in FIGS. 14A and 14B, the enhanced expression of SYVN1 was dose-dependent. Furthermore, a dose-dependent Mnk1 depletion and suppression of its down-stream target, peIF4E and cyclin D1 was also observed. See FIG. 14B. Further studies of Mnk1 and Mnk2 with VNLG-152R, as well as deuterated analogs thereof, such as Compounds 4, 8 and 9, are potential future targets of study.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

1. Vorobiof D. A. Recent advances in the medical treatment of breast cancer. F1000Res. 2016; 5:2786.
2. Pareja F., Geyer F. C., Marchio C., Burke K. A., Weigelt B., Reis-Filho J. S. Triple-negative breast cancer: The importance of molecular and histologic subtyping, and recognition of low-grade variants. NPJ Breast Cancer. 2016; 2:16036.
3. Jemal A, Ward E M, Johnson C J, Cronin K A, Ma J, Ryerson B, Mariotto A, Lake A J, Wilson R, Sherman R L et al. (2017) Annual Report to the Nation on the Status of Cancer, 1975-2014, featuring survival. J Natl Cancer Inst 109.
4. Saad F & Fizazi K (2015) Androgen deprivation therapy and secondary hormone therapy in the management of hormone-sensitive and castration-resistant prostate cancer. Urology 86, 852-861.
5. Siddiqui, N.; Sonenberg, N. Signalling to eIF4E in cancer. Biochem. Soc. Trans. 2015, 43, 763-772.
6. Sonenberg, N. eIF4E, the mRNA cap-binding protein: From basic discovery to translational research. Biochem. Cell Biol. 2008, 86, 178-183.
7. Wendel, H. G.; Silva, R. L.; Malina, A.; Mills, JR.; Zhu, H.; Ueda, T.; Watanabe-Fukunaga, R.; Fukunaga, R.; Teruya-Feldstein, J.; Pelletier, J.; et al. Dissecting eIF4E action in tumorigenesis. Genes Dev. 2007, 21, 3232-3237.
8. Lachance, P. E.; Miron, M.; Raught, B.; Sonenberg, N.; Lasko, P. Phosphorylation of eukaryotic translation initiation factor 4E is critical for growth. Mol. Cell. Biol 2002, 22, 1656-1663.
9. Ueda, T.; Watanabe-Fukunaga, R.; Fukuyama, H.; Nagata, S.; Fukunaga, R. Mnk2 and Mnk1 are essential for constitutive and inducible phosphorylation of eukaryotic initiation factor 4E but not for cell growth or development. Mol. Cell. Biol. 2004, 24, 6539-6549.
10. Soni A., Akcakanat A., Singh G., Luyimbazi D., Zheng Y., Kim D., Gonzalez-Angulo A., Meric-Bernstam F. eIF4E knockdown decreases breast cancer cell growth without activating Akt signaling. Mol. Cancer Ther. 2008; 7:1782-1788.
11. Li Z., Sun Y., Qu M., Wan H., Cai F., Zhang P. Inhibiting the MNK-eIF4E-beta-catenin axis increases the responsiveness of aggressive breast cancer cells to chemotherapy. Oncotarget. 2017; 8:2906-2915.
12. Wheater M. J., Johnson P. W., Blaydes J. P. The role of MNK proteins and eIF4E phosphorylation in breast cancer cell proliferation and survival. Cancer Biol. Ther. 2010; 10:728-735.
13. Flowers A., Chu Q. D., Panu L., Meschonat C., Caldito G., Lowery-Nordberg M., Li B. D. Eukaryotic initiation factor 4E overexpression in triple-negative breast cancer predicts a worse outcome. Surgery. 2009; 146:220-226.
14. Nathan C. O., Carter P., Liu L., Li B. D., Abreo F., Tudor A., Zimmer S. G., De Benedetti A. Elevated expression of eIF4E and FGF-2 isoforms during vascularization of breast carcinomas. Oncogene. 1997; 15:1087-1094.
15. Robichaud N., Del Rincon S. V., Huor B., Alain T., Petruccelli L. A., Hearnden J., Goncalves C., Grotegut S., Spruck C. H., Furic L., et al. Phosphorylation of eIF4E promotes EMT and metastasis via translational control of SNAIL and MMP-3. Oncogene. 2014.
16. Byrnes K., White S., Chu Q., Meschonat C., Yu H., Johnson L. W., Debenedetti A., Abreo F., Turnage R. H., McDonald J. C., et al. High eIF4E, VEGF, and microvessel density in stage I to III breast cancer. Ann. Surg. 2006; 243:684-690.
17. Li B. D., Liu L., Dawson M., De Benedetti A. Overexpression of eukaryotic initiation factor 4E (eIF4E) in breast carcinoma. Cancer. 1997; 79:2385-2390.
18. Li B. D., McDonald J. C., Nassar R., De Benedetti A. Clinical outcome in stage I to III breast carcinoma and eIF4E overexpression. Ann. Surg. 1998; 227:756-761; discussion 761-763.
19. Lineham E., Tizzard G. J., Coles S. J., Spencer J., Morley S. J. Synergistic effects of inhibiting the MNK-eIF4E and PI3K/AKT/mTOR pathways on cell migration in MDA-MB-231 cells. Oncotarget. 2018; 9:14148-14159.
20. Pinto-Diez C., Garcia-Recio E. M., Perez-Morgado M. I., Garcia-Hernandez M., Sanz-Criado L., Sacristan S., Toledo-Lobo M. V., Perez-Mies B., Esteban-Rodriguez I., Pascual A., et al. Increased expression of MNK1b, the spliced isoform of MNK1, predicts poor prognosis and is associated with triple-negative breast cancer. Oncotarget. 2018; 9:13501-13516.
21. Reich S. H., Sprengeler P. A., Chiang G. G., Appleman J R., Chen J., Clarine J., Eam B., Ernst J. T., Han Q., Goel V. K., et al. Structure-based Design of Pyridone-Aminal eFT508 Targeting Dysregulated Translation by Selective Mitogen-activated Protein Kinase Interacting Kinases 1 and 2 (MNK1/2) Inhibition. J. Med. Chem. 2018; 61:3516-3540.
22. Thompson E W, Newgreen D F & Tarin D (2005) Carcinoma invasion and metastasis: a role for epithelial-mesenchymal transition?Cancer Res 65, 5991-5995; discussion 5995.
23. Thiery J P (2002) Epithelial-mesenchymal transitions in tumour progression. Nat Rev Cancer 2, 442-454.
24. Fan S., Li Y., Yue P., Khuri F. R., Sun S. Y. The eIF4E/eIF4G interaction inhibitor 4EGI-1 augments TRAIL-mediated apoptosis through c-FLIP Down-regulation and DR5 induction independent of inhibition of cap-dependent protein translation. Neoplasia. 2010; 12:346-356.
25. Grant C M & Kyprianou N (2013) Epithelial mesenchymal transition (EMT) in prostate growth and tumor progression. Transl Androl Urol 2, 202-211.
26. Montanari M, Rossetti S, Cavaliere C, D'Aniello C, Malzone M G, Vanacore D, Di Franco R, La Mantia E, Iovane G, Piscitelli R et al. (2017) Epithelial-mesenchymal transition in prostate cancer: an overview. Oncotarget 8, 35376-35389.
27. Zhu M L & Kyprianou N (2010) Role of androgens and the androgen receptor in epithelial-mesenchymal transition and invasion of prostate cancer cells. FASEB J 24, 769-777.
28. Matuszak E A & Kyprianou N (2011) Androgen regulation of epithelial-mesenchymal transition in prostate tumorigenesis. Expert Rev Endocrinol Metab 6, 469-482.
29. Robichaud N, del Rincon S V, Huor B, Alain T, Petruccelli L A, Hearnden J, Goncalves C, Grotegut S, Spruck C H, Furic L et al. (2015) Phosphorylation of eIF4E promotes EMT and metastasis via translational control of SNAIL and MMP-3. Oncogene 34, 2032-2042.
30. Wang Y, Shi J, Chai K, Ying X & Zhou B P (2013) The role of Snail in EMT and tumorigenesis. Curr Cancer Drug Targets 13, 963-972.
31. Chen A, Beetham H, Black M A, Priya R, Telford B J, Guest J, Wiggins G A, Godwin T D, Yap A S & Guilford P J (2014) E-cadherin loss alters cytoskeletal organization and adhesion in non-malignant breast cells but is insufficient to induce an epithelial-mesenchymal transition. BMC Cancer 14, 552.
32. Fink K & Boratynski J (2012) The role of metalloproteinases in modification of extracellular matrix in invasive tumor growth, metastasis and angiogenesis. Postepy Hig Med Dosw (Online) 66, 609-628.
33. Brown G T & Murray G I (2015) Current mechanistic insights into the roles of matrix metalloproteinases in tumour invasion and metastasis. J Pathol 237, 273-281.
34. Aktas B H; Qiao Y; Ozdelen E; Schubert R; Sevinc S; Harbinski F; Grubissich L; Singer S; Halperin J A Small-molecule targeting of translation initiation for cancer therapy. Oncotarget 2013, 4 (10), 1606-1617.
35. Bitterman P B; Polunovsky V A Attacking a nexus of the oncogenic circuitry by reversing aberrant eIF4F-mediated translation. Mol. Cancer Ther 2012, 11 (5), 1051-1061.
36. Bitterman P B; Polunovsky V A Translational control of cell fate: from integration of environmental signals to breaching anticancer defense. Cell Cycle 2012, 11 (6), 1097-1107.
37. Carroll M; Borden K L The oncogene eIF4E: using biochemical insights to target cancer. J. Interferon Cytokine Res 2013, 33 (5), 227-238.
38. Diab S; Kumarasiri M; Yu M; Teo T; Proud C; Milne R; Wang S MAP kinase-interacting kinases-emerging targets against cancer. Chem. Biol 2014, 21 (4), 441-452.
39. Bhat M., Robichaud N., Hulea L., Sonenberg N., Pelletier J., Topisirovic I. Targeting the translation machinery in cancer. Nat. Rev. Drug Discov. 2015; 14:261.
40. Pelletier J., Graff J., Ruggero D., Sonenberg N. Targeting the eIF4F translation initiation complex: A critical nexus for cancer development. Cancer Res. 2015; 75:250-263. doi: 10.1158/0008-5472.CAN-14-2789.
41. Pettersson F., Del Rincon S. V., Emond A., Huor B., Ngan E., Ng J., Dobocan M. C., Siegel P. M., Miller W. H., Jr. Genetic and pharmacologic inhibition of eIF4E reduces breast cancer cell migration, invasion, and metastasis. Cancer Res. 2015; 75:1102-1112.
42. Pettersson F., Del Rincon S. V., Miller W. H., Jr. Eukaryotic translation initiation factor 4E as a novel therapeutic target in hematological malignancies and beyond. Expert Opin. Ther. Targets. 2014; 18:1035-1048.
43. Pettersson F., Yau C., Dobocan M. C., Culjkovic-Kraljacic B., Retrouvey H., Puckett R., Flores L. M., Krop I. E., Rousseau C., Cocolakis E., et al. Ribavirin treatment effects on breast cancers overexpressing eIF4E, a biomarker with prognostic specificity for luminal B-type breast cancer. Clin. Cancer Res. 2011; 17:2874-2884.
44. Ramamurthy V. P., Ramalingam S., Kwegyir-Afful A. K., Hussain A., Njar V. C. Targeting of protein translation as a new treatment paradigm for prostate cancer. Curr. Opin. Oncol. 2017; 29:210-220.
45. Brown M. C., Gromeier M. MNK Controls mTORC1: Substrate Association through Regulation of TELO2 Binding with mTORC1. Cell Rep. 2017; 18:1444-1457.
46. Brown M. C., Gromeier M. MNK inversely regulates TELO2 vs. DEPTOR to control mTORC1 signaling. Mol. Cell. Oncol. 2017; 4:e1306010.
47. Ramamurthy, V. P., Ramalingam, S., Gediya, L., Kwegyir-Afful, A. K., Njar, V. C., Simultaneous targeting of androgen receptor (AR) and MAPK-interacting kinases (MNKs) by novel retinamides inhibits growth of human prostate cancer cell lines, Oncotarget, 2015, 6(5), 3195-3210.
48. Ramalingam, S., Gediya, L., Kwegyir-Afful, A. K., Ramamurthy, V. P., Purushottamachar, P., Mbatia H., Njar, V. C., First MNKs degrading agents block phosphorylation of eIF4E, induce apoptosis, inhibit cell growth, migration and invasion in triple negative and Her2-overexpressing breast cancer cell lines, Oncotarget, 2014, 5(2), 530-543.
49. Gediya L K, Belosay A, Khandelwal A, Purushottamachar P, Njar V C. Improved synthesis of histone deacetylase inhibitors (HDIs) (MS-275 and CI-994) and inhibitory effects of HDIs alone or in combination with RAMBAs or retinoids on growth of human LNCaP prostate cancer cells and tumor xenografts. Bioorg Med Chem. 2008; 16(6):3352-3360.
50. Njar V C, Gediya L, Purushottamachar P, Chopra P, Vasaitis T S, Khandelwal A, Mehta J, Huynh C, Belosay A, Patel J. Retinoic acid metabolism blocking agents (RAMBAs) for treatment of cancer and dermatological diseases. Bioorg Med Chem. 2006; 14(13):4323-4340.
51. Patel J B, Huynh C K, Handratta V D, Gediya L K, Brodie A M, Goloubeva O G, Clement 00, Nanne I P, Soprano D R, Njar V C. Novel retinoic acid metabolism blocking agents endowed with multiple biological activities are efficient growth inhibitors of human breast and prostate cancer cells in vitro and a human breast tumor xenograft in nude mice. J Med Chem. 2004; 47(27):6716-6729.
52. Patel J B, Khandelwal A, Chopra P, Handratta V D, Njar V C. Murine toxicology and pharmacokinetics of novel retinoic acid metabolism blocking agents. Cancer Chemother Pharmacol. 2007; 60(6):899-905.
53. Lotan et al., Retinoids and apoptosis, implications for cancer chemoprevention and therapy, Journal of National Cancer Institute, 87: 1655-1657, 1995.
54. Mangelsdorf et al., The nuclear receptor superfamily, the second decade, Cell, 83: 835-839, 1995.
55. Ramamurthy V. P., Ramalingam S., Gediya L. K., Njar V. C. O. The retinamide VNLG-152 inhibits f-AR/AR-V7 and MNK-eIF4E signaling pathways to suppress EMT and castration-resistant prostate cancer xenograft growth. FEBS J. 2018; 285:1051-1063.
56. Ramalingam, S.; Ramamurthy, V. P.; Gediya, L. K.; Murigi, F. N.; Purushottamachar, P.; Huang, W.; Choi, E. Y.; Zhang, Y.; Vasaitis, T. S.; Kane, M. A.; et al. The Novel Mnk1/2 Degrader and Apoptosis Inducer VNLG-152 Potently Inhibits TNBC Tumor Growth and Metastasis. Cancers (Basel) 2019, 11 (3).
57. Cargnin, S.; Serafini, M.; Pirali, T. A primer of deuterium in drug design. Future Med Chem 2019, 11 (16), 2039-2042.
58. Gant, T. G. Using deuterium in drug discovery: leaving the label in the drug. Journal of medicinal chemistry 2014, 57 (9), 3595-3611. Howland, R. H. Deuterated Drugs. J Psychosoc Nurs Ment Health Serv 2015, 53 (9), 13-16. Pirali, T.; Serafini, M.; Cargnin, S.; Genazzani, A. A. Applications of Deuterium in Medicinal Chemistry. Journal of medicinal chemistry 2019, 62 (11), 5276-5297.
59. Russak, E. M.; Bednarczyk, E. M. Impact of Deuterium Substitution on the Pharmacokinetics of Pharmaceuticals. Ann Pharmacother 2019, 53 (2), 211-216.
60. DeWitt, S. H.; Maryanoff, B. E. Deuterated Drug Molecules: Focus on FDA-Approved DeutetrabenazinePublished as part of the Biochemistry series “Biochemistry to Bedside”. Biochemistry 2018, 57 (5), 472-473.
61. Schmidt, C. First deuterated drug approved. Nat Biotechnol 2017, 35 (6), 493-494.
62. Tung, R. D. Deuterium medicinal chemistry comes of age. Future Med Chem 2016, 8 (5), 491-494.
63. Gunaydin, H.; Altman, M. D.; Ellis, J. M.; Fuller, P.; Johnson, S. A.; Lahue, B.; Lapointe, B. Strategy for Extending Half-life in Drug Design and Its Significance. ACS Med Chem Lett 2018, 9 (6), 528-533.
64. Purushottamachar, P.; Godbole, A. M.; Gediya, L. K.; Martin, M. S.; Vasaitis, T. S.; Kwegyir-Afful, A. K.; Ramalingam, S.; Ates-Alagoz, Z.; Njar, V. C. Systematic structure modifications of multitarget prostate cancer drug candidate galeterone to produce novel androgen receptor down-regulating agents as an approach to treatment of advanced prostate cancer. Journal of medicinal chemistry 2013, 56 (12), 4880-4898.
65. Nagilla, R.; Nord, M.; McAtee, J. J.; Jolivette, L. J. Cassette dosing for pharmacokinetic screening in drug discovery: comparison of clearance, volume of distribution, half-life, mean residence time, and oral bioavailability obtained by cassette and discrete dosing in rats. J Pharm Sci 2011, 100 (9), 3862-3874. Smith, N. F.; Raynaud, F. I.; Workman, P. The application of cassette dosing for pharmacokinetic screening in small-molecule cancer drug discovery. Molecular cancer therapeutics 2007, 6 (2), 428-440. Sun, M.; Xiao, H.; Hong, H.; Zhang, A.; Zhang, Y.; Liu, Y.; Zhu, L.; Kung, H. F.; Qiao, J. Rapid screening of nine unradiolabeled candidate compounds as PET brain imaging agents using cassette-wave microdosing and LC-MS/MS. J Chromatogr B Analyt Technol Biomed Life Sci 2019, 1121, 28-38. Watanabe, T.; Schulz, D.; Morisseau, C.; Hammock, B. D. High-throughput pharmacokinetic method: cassette dosing in mice associated with minuscule serial bleedings and LC/MS/MS analysis. Anal Chim Acta 2006, 559 (1), 37-44.
66. Sharma, R.; Strelevitz, T. J.; Gao, H.; Clark, A. J.; Schildknegt, K.; Obach, R. S.; Ripp, S. L.; Spracklin, D. K.; Tremaine, L. M.; Vaz, A. D. Deuterium isotope effects on drug pharmacokinetics. I. System-dependent effects of specific deuteration with aldehyde oxidase cleared drugs. Drug Metab Dispos 2012, 40 (3), 625-634.
67. Uttamsingh, V.; Gallegos, R.; Liu, J. F.; Harbeson, S. L.; Bridson, G. W.; Cheng, C.; Wells, D. S.; Graham, P. B.; Zelle, R.; Tung, R. Altering metabolic profiles of drugs by precision deuteration: reducing mechanism-based inhibition of CYP2D₆ by paroxetine. J Pharmacol Exp Ther 2015, 354 (1), 43-54.
68. Dunsaed, C. B.; Dornish, J. M.; Pettersen, E. O. The bioavailability and dose dependency of the deuterated anti-tumour agent 4,6-benzylidene-dl-D-glucose in mice and rats. Cancer Chemother Pharmacol 1995, 35 (6), 464-470.
69. Phase 3 Study of ALK-001 in Geographic Atrophy (SAGA), available at https://clinicaltrials.gov/ct2/show/NCT03845582 (last visited Mar. 2, 2022); U.S. Pat. No. 9,486,432 to Washington.
70. Huang W, Yu J, Jones J W, Carter C L, Jackson I L, Vujaskovic Z, MacVittie T J, Kane M A: Acute Proteomic Changes in the Lung After WTLI in a Mouse Model: Identification of Potential Initiating Events for Delayed Effects of Acute Radiation Exposure. Health Phys 2019, 116:503-15.
71. Huang W, Yu J, Jones J W, Carter C L, Pierzchalski K, Tudor G, Booth C, MacVittie T J, Kane M A: Proteomic Evaluation of the Acute Radiation Syndrome of the Gastrointestinal Tract in a Murine Total-body Irradiation Model. Health Phys 2019, 116:516-28.

1. A compound of formula (A) 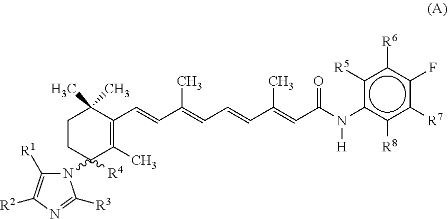
or a pharmaceutically acceptable salt thereof,
wherein each of R¹ to R⁸ is independent H or D, and at least one of R¹ to R⁸ is D.

2. The compound of claim 1, wherein R⁴ and at least one of R⁶ to R⁸ are D.

3. The compound of claim 1, which is at least one selected from the group consisting of: 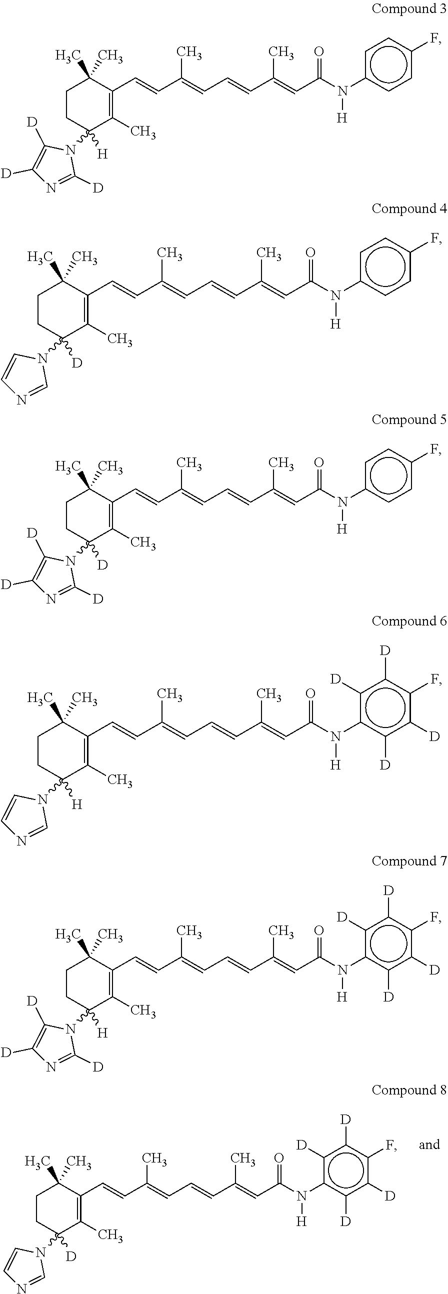 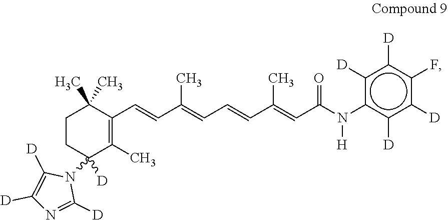
or a pharmaceutically acceptable salt thereof.

4. The compound of claim 1, which is at least one selected from the group consisting of: 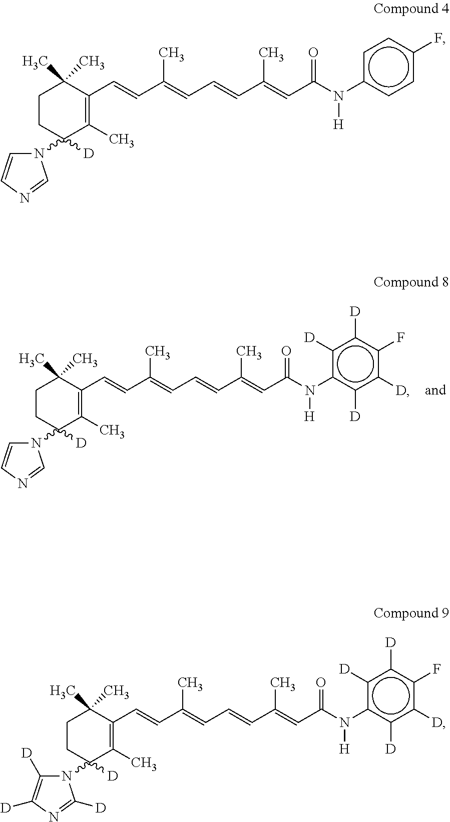
or a pharmaceutically acceptable salt thereof.

5. A pharmaceutical composition comprising the compound of claim 1 or a pharmaceutically acceptable salt thereof, and a pharmaceutically acceptable carrier.

6. The pharmaceutical composition of claim 5, wherein the compound is at least one selected from the group consisting of: 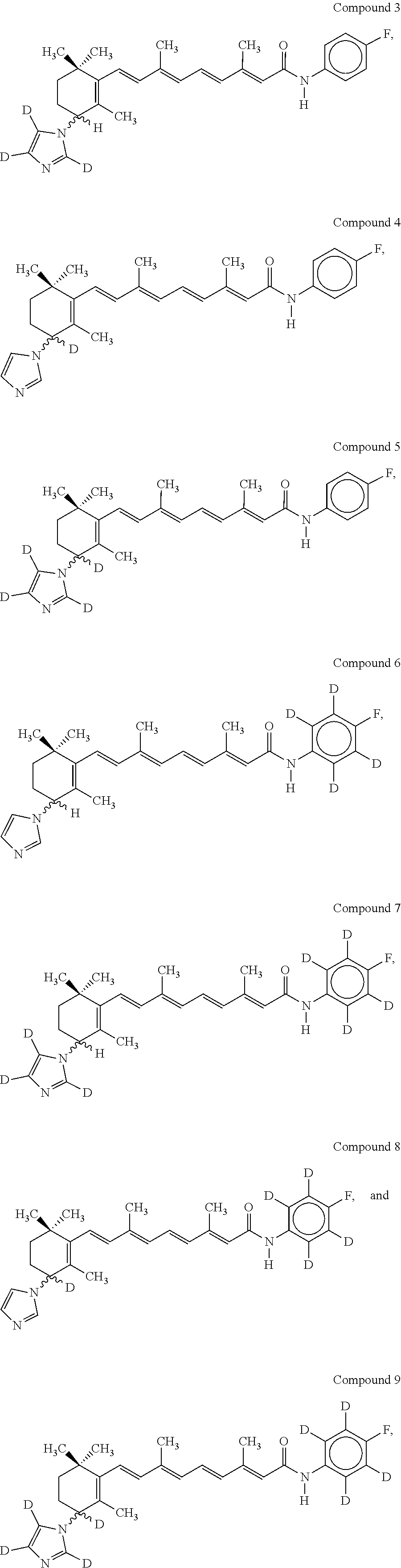
or a pharmaceutically acceptable salt thereof.

7. The pharmaceutical composition according to claim 5, wherein the compound is at least one selected from the group consisting of: 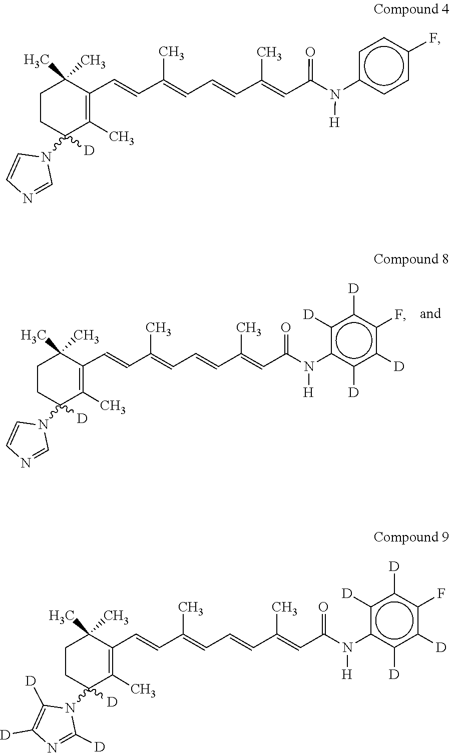
or a pharmaceutically acceptable salt thereof.

8. The pharmaceutical composition according to claim 5, wherein the composition is formulated for oral administration.

9. A method of treating a disease, comprising administering a therapeutically effective amount of the compound of claim 1 or a pharmaceutically acceptable salt thereof to a subject in need thereof,
wherein the disease is one of breast cancer, prostate cancer, bladder, cancer, pancreatic cancer, hepatocellular carcinoma, benign prostatic hyperplasia, Kennedy's disease, and hematologic cancers.

10. The method according to claim 9, wherein the compound is at least one selected from the group consisting of: 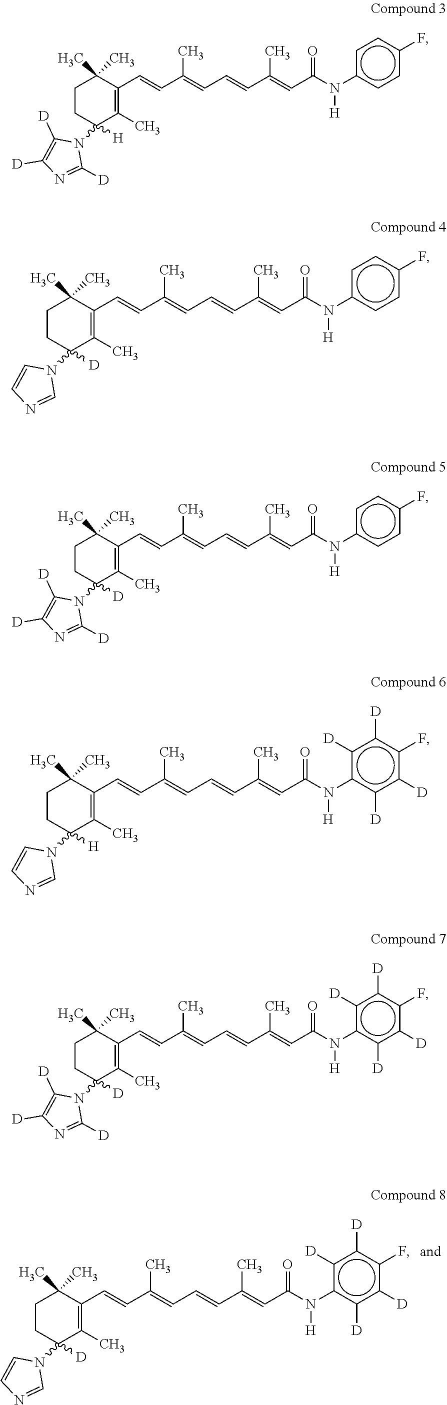 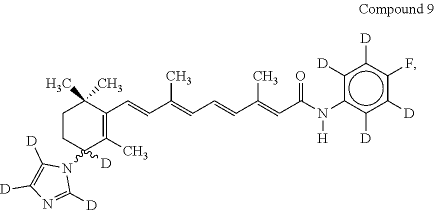
or a pharmaceutically acceptable salt thereof.

11. The method of claim 9, wherein the compound is at least one selected from the group consisting of: 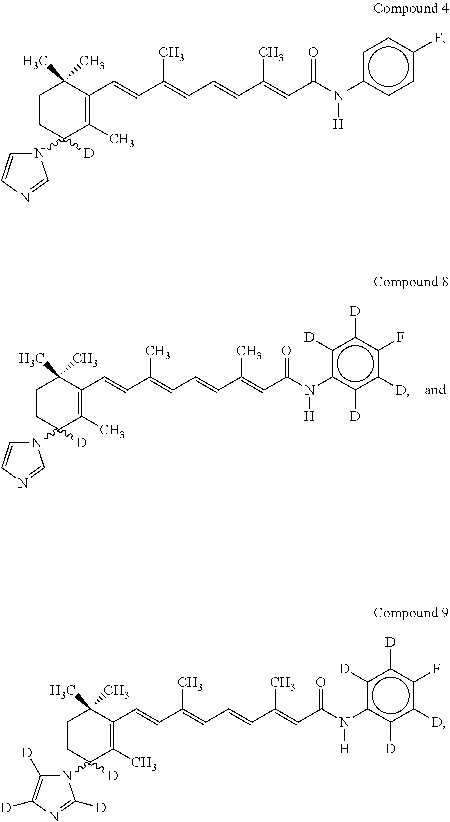
or a pharmaceutically acceptable salt thereof.

12. The method of claim 9, wherein the disease is breast cancer.

13. The method of claim 12, wherein the breast cancer is triple negative breast cancer.

14. The method of claim 9, wherein the compound is administered orally.

15. A method of preparing a compound of formula (A): 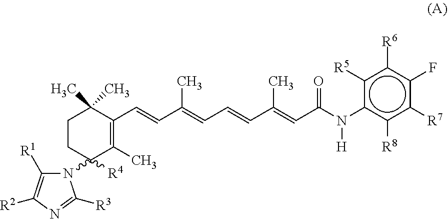
or a salt thereof, the method comprising converting a compound of formula (B): 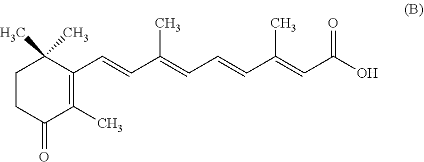
or a salt thereof, into the compound of formula (A) or the salt thereof,
wherein each of R¹ to R⁸ is independent H or D, and at least one of R¹ to R⁸ is D.

16. The method according to claim 15, wherein the converting of the compound of formula (B) comprises:
converting the compound of formula (B) into a compound of formula (C) or a salt thereof or a compound of formula (D) or a salt thereof:

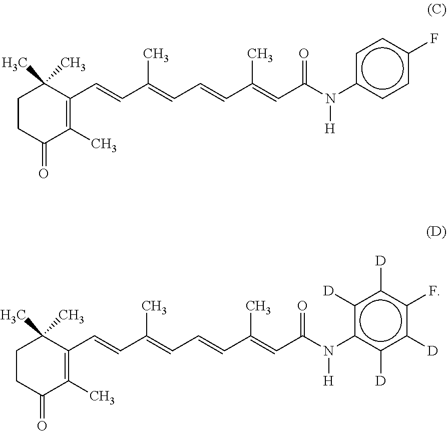

17. The method according to claim 16, further comprising converting the compound of formula (C) or the salt thereof into a compound of formula (E) or a salt thereof or a compound of formula (F) or a salt thereof:

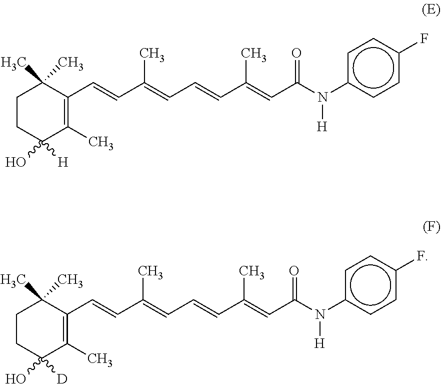

18. The method according to claim 16, further comprising converting the compound of formula (D) or a salt thereof into a compound of formula (G) or a salt thereof or a compound of formula (H) or a salt thereof:

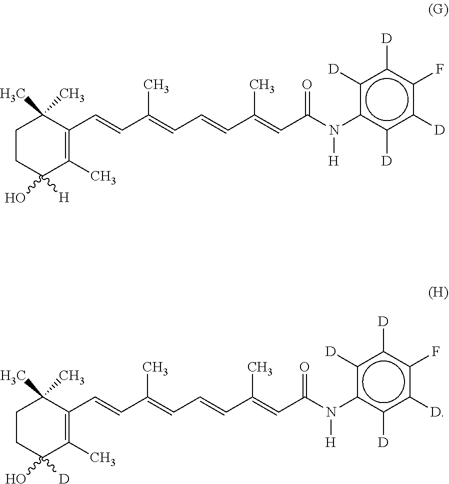

19. A method of preparing a compound of formula (I): 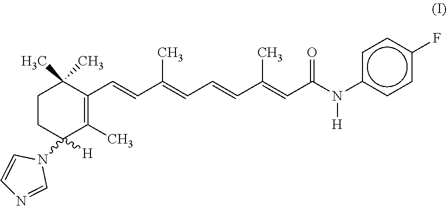
or a salt thereof, the method comprising converting a compound of formula (B) into the compound of formula (I):

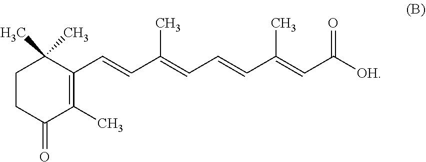

20. The method according to claim 19, wherein the converting of the compound of formula (B) comprises:
converting the compound of formula (B) into a compound of formula (C): 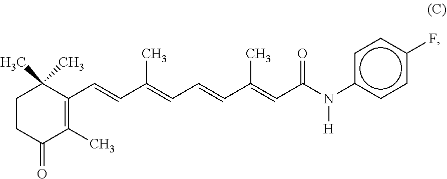
converting the compound of formula (C) into a compound of formula (E): 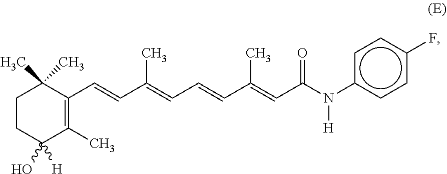
converting the compound of formula (E) into the compound of formula (I).
